(19) United States
(12) Patent Application Publication
(10) Pub. No.: US 2022/0031831 A1
(43) Pub. Date: Feb. 3, 2022

(54) IMMUNOGENIC COMPOSITIONS FOR AFRICAN SWINE FEVER VIRUS

(71) Applicant: Kansas State University Research Foundation, Manhattan, KS (US)

(72) Inventors: Waithaka Mwangi, Manhattan, KS (US); Shehnaz Lokhandwala, Manhattan, KS (US)

(21) Appl. No.: 17/309,272

(22) PCT Filed: Nov. 13, 2019
(86) PCT No.: PCT/US19/61207

§ 371 Date: May 13, 2021

Related U.S. Application Data
(60) Provisional application No. 62/767,594, filed on Nov. 15, 2018.

(51) Int. Cl. A61K 39/12; C12N 15/86; A61P 31/20

(52) U.S. Cl. CPC A61K 39/12; A61P 31/20; C12N 15/86

(57) ABSTRACT

The present disclosure provides immunogenic compositions or vaccines against African Swine Fever Virus (ASFV), methods of making and using such immunogenic compositions or vaccines, and methods of administering such immunogenic compositions or vaccines. In some forms, a plurality of ASFV antigens are combined together into a live-vectored multivalent immunogenic composition. In some forms, the live-vectored multivalent immunogenic composition is a multicistronic expression cassette.

### GOVERNMENT LICENSE RIGHTS

This invention was made with government support under USDA-NIFA 2016-67015-25041 awarded by United States Department of Agriculture. The government has certain rights in the invention.

### SEQUENCE LISTING

This application contains a sequence listing in paper format and in computer readable format, the teachings and content of which are hereby incorporated by reference.

### BACKGROUND

African Swine Fever Virus (“ASFV”) is a highly contagious hemorrhagic disease of pigs that resembles classical swine fever in its presentation of clinical signs and lesions. ASFV is enzootic in many African countries as well as in Sardinia. Beginning in 2007, ASFV spread into domestic and wild pig populations in western and southern Russia. This spread has continued into Europe and threatens pig populations throughout the continent.

ASFV is a large, enveloped, double-stranded DNA virus that replicates primarily in cells of the mononuclear phagocytic system. It is a member of the Asfarviridae family. Genotypes of ASFV are differentiated by sequence analysis of the genomes of viruses obtained from different geographic areas. The virus is highly resistant to a wide pH range and to a freeze/thaw cycle and can remain infectious for many months at room temperature or when stored at 4 degrees C. Virus in body fluids and serum is inactivated in 30 min at 60 degrees C., but virus in unprocessed pig meat can be inactivated by heating to 70 degrees for 30 minutes. Although ASFV can be adapted to grow in cells from different species, it does not replicate readily in any species other than swine.

Peracute, acute, subacute, and chronic forms of ASFV occur and mortality rates vary from 0 to 100%. Acute disease is characterized by a short incubation period of 3-7 days, followed by high fever (up to 42 degrees C.) and death in 5-10 days. Clinical signs include loss of appetite; depression; recumbancy; hyperemia of the skin of the ears, abdomen, and legs; respiratory distress; vomiting; bleeding from the nose or rectum; diarrhea; and abortion. Lesions are evidenced in the lymph nodes, kidneys, heart, as well as other organs. Some isolates produce an enlarged and friable spleen; straw-colored or blood-stained fluid in pleural, pericardial, and peritoneal cavities; or edema and congestion of the lungs. Chronic disease is characterized by emaciation, swollen joints, and respiratory problems. Because ASFV cannot be distinguished from classical swine fever by either clinical or postmortem examination, infection must be confirmed by PCR, ELISA, or indirect immunofluorescence.

There is no treatment or prophylactic for ASFV. Successful eradication involves the slaughter and disposal of all animals on the premises.

What is needed is a composition and accompanying methods of making and administering the composition that reduces the severity of or incidence of clinical and postmortem signs of ASFV infection.

### SUMMARY OF THE DISCLOSURE

The present disclosure overcomes the problems inherent in the art and provides immunogenic compositions or vaccines against ASFV, methods of making and using such immunogenic compositions or vaccines, and methods of administering such immunogenic compositions or vaccines.

In one aspect, the present disclosure generally provides an efficacious African Swine Fever Virus (ASFV) immunogenic composition or vaccine.

In another aspect, the present disclosure generally provides methods for making and/or producing an efficacious African Swine Fever Virus immunogenic composition or vaccine.

In another aspect, the present disclosure generally provides an immunogenic composition or vaccine that reduces the severity of or the incidence of infection by ASFV.

In another aspect, the present disclosure generally provides methods for reducing the incidence and/or severity of clinical and postmortem signs of ASFV infection.

In another aspect, the present disclosure generally provides a compatible DIVA Lateral Flow Device (LFD) for rapid diagnosis.

In some forms of all aspects, the immunogenic composition comprises at least one ASFV antigen. Preferably, the antigen is conserved among ASFV isolates. One preferred source for conserved antigens is from the Georgia 2007/1 ASFV isolate. In preferred forms, a plurality of antigens are combined together into a live-vectored multivalent immunogenic composition. In some forms, the live-vectored multivalent immunogenic composition is a multicistronic expression cassette. In some forms, the multicistronic expression cassette includes 1, 2, 3, 4, 5, 6, 7, 8, 9, 10, 11, 12, 13, 14, 15, 16, 17, 18, 19, 20, or more individual antigens. In some forms, the multicistronic cassette includes an insert of at least 1 kBp, 2 kBp, 3 kBp, 4 kBp, 5 kBp, 6 kBp, 7 kBp, or 8 kBp. Some preferred antigens are selected from the group consisting of sequences having at least 80%, more preferably 85%, still more preferably 90%, even more preferably 91%, 92%, 93%, 94%, 95%, 96%, 97%, 98%, 99%, or 100% sequence identity or sequence homology with a sequence selected from SEQ ID NOS. 1-101. Any one or number of SEQ ID NOS. 1-101 can be combined together to form a multicistronic expression cassette. In other words, the individual sequences can be combined together in any order and with any number of individual sequences being combined. Some preferred multicistronic expression cassettes are provided as SEQ ID NOS. 102-131. As can be appreciated, the antigens forming each expression cassette can vary in their sequence identity to the individual sequences as described above, therefore, the overall sequence identity or homology required for an expression cassette is at least 80%, more preferably 85%, still more preferably 90%, even more preferably 91%, 92%, 93%, 94%, 95%, 96%, 97%, 98%, 99%, or 100% with a sequence selected from SEQ ID NOS. 102-131. Further, the individual sequences forming a multicistronic expression cassette can appear in any order. For example, SEQ ID NO. 104 includes the antigens from SEQ ID NOS. 2, 3, and 4. In SEQ ID NO. 104, these appear in numerical order 2, 3, and 4, respectively. However, they can be in any order such as 4, 3, and 2; or 3, 2, and 4, or 2, 4, and 3. In some forms, the individual sequences are fused or included in-frame to create a chimeric antigenic sequence. In some forms, a self-cleaving peptide linker is included between the different antigens forming the multivalent composition.

In some forms of a live-vectored multivalent immunogenic composition of the disclosure, the multicistronic expression cassette utilizes a replication incompetent vector, such as a recombinant adenovirus. In other forms, the multicistronic expression cassette utilizes a single cycle replicon virus, preferably a single cycle replicon adenovirus, as the vector. In still other forms, the multicistronic cassette utilizes an attenuated bovine parainfluenza virus type 3 genotype c (BPIV3c) as the vector. In addition to inducing strong cytotoxic T lymphocyte (CTL) immune responses, compositions of the disclosure generate IFN-γ and antibody responses. In still other forms, a baculovirus or lentivirus vector is utilized.

In another aspect, the present disclosure generally provides nucleic acid sequences for expressing any of SEQ ID NOS. 1-131 or sequences having at least 80%, more preferably 85%, still more preferably 90%, even more preferably 91%, 92%, 93%, 94%, 95%, 96%, 97%, 98%, 99%, or 100% sequence identity or homology with any of SEQ ID NOS. 1-131. Such nucleic acid sequences can be placed in a vector for expression wherein the resultant construct is administered to a subject as described in this disclosure or the expressed antigens are recovered and used in accordance with methods of this disclosure or administered in accordance with methods of this disclosure.

In another aspect of the present disclosure, a fragment of a nucleic acid sequence or expressed antigen is provided. In preferred forms, the fragments comprise a portion of any of SEQ ID NOS. 1-131, or the proteins expressed therefrom. In preferred forms, a nucleic acid fragment of any one or more of SEQ ID NOS. 1-131 includes at least 15, 21, 30, 45, 60, 75, 99, 120, 150, or 180 consecutive nucleotides of the specific sequence from SEQ ID NOS. 1-131 or sequences having at least 80%, more preferably 85%, still more preferably 90%, even more preferably 91%, 92%, 93%, 94%, 95%, 96%, 97%, 98%, 99%, or 100% sequence identity or homology with any of SEQ ID NOS. 1-131. Similarly, a fragment of an antigen expressed by any one of SEQ ID NOS. 1-131 or at least 80%, more preferably 85%, still more preferably 90%, even more preferably 91%, 92%, 93%, 94%, 95%, 96%, 97%, 98%, 99%, or 100% sequence identity or homology with any of SEQ ID NOS. 1-131 will preferably include at least 5, 7, 10, 15, 20, 25, 33, 40, 50, or 60 consecutive amino acids from the specific expressed sequence.

Knowledge gleaned from previous and ongoing ASFV vaccine studies suggests that an efficacious subunit vaccine will preferably include a system capable of cytosolic antigen expression and amplification for effective priming and expansion of cytotoxic T lymphocytes (CTLs) which are required to eliminate infected cells. In some forms, a preferred system includes a replicon system. To this end, we have identified and validated promising conserved ASFV (Georgia 2007/1) CTL antigens and used the genes encoding these antigens to develop a prototype immunogenic composition or vaccine, designated SC-Ad-ASFV, composed of next generation single-cycle replicon adenovirus (SC-Ad) encoding multicistronic expression cassettes that incorporate conserved African Swine Fever Virus (ASFV) antigens. The ASFV Georgia 2007/1 isolate was selected because it is highly virulent and epidemiologically relevant. A chimeric antigen, designated KP1712, was also generated for development of a compatible DIVA lateral flow-based highly sensitive diagnostic tool.

The composition according to the disclosure may be administered or applied systemically through an intravenous, intravascular, intramuscular, intranasal, intraarterial, intraperitoneal, oral, subcutaneous, transdermal, or intrathecal route. Preferably, the composition is administered or applied orally or intramuscularly. For an oral administration, an edible bait is most preferred as this will also permit immunization of pigs, wild boars, and feral pigs. Depending on the desired duration and effectiveness of the treatment, the compositions according to the disclosure may be administered once or several times, also intermittently, for instance on a daily basis for several days, weeks or months, and in different dosages.

Selection of Novel Vaccine Candidate Antigens

Since peptide presentation by Swine Leukocyte Antigen I (SLA I) is required to prime and expand CTLs, a strategy that utilizes an algorithm based on SLA I-peptide binding is expected to identify putative candidate antigens based on the presence of peptide motifs predicted to bind with high affinity to defined SLA I molecules. All the conserved ASFV (Georgia 2007/1) open-reading frames (ORFs) were screened for the presence of peptide motifs that bind strongly to 75 defined SLA I alleles and identified novel targets containing putative CTL epitopes (found on the web at cbs.dtu.dk/services/NetMHCpan/). The targets were ranked based on the number of putative CTL epitopes and binding affinity (FIG. 1). The targets were validated using ASFV convalescent serum followed by in vitro confirmation of the SLA class I-binding motifs using peptides (FIGS. 2 and 3).

One hundred ORFs were selected (see list below) and used to design multicistronic expression cassettes separated by an efficient self-cleaving 2A peptide linker (FIG. 2). The multicistronic expression cassettes were modified to add, in-frame, a HA-tag at the N-termini and a FLAG-tag at the C-termini (FIG. 2A). The amino acid sequences of the multicistronic cassettes were used to design synthetic genes codon-optimized for protein expression in swine cells. Recombinant DNA and SC-Ad expression constructs were generated using the synthetic genes (FIG. 2B). Protein expression by the constructs was evaluated by immunocytometric analysis using anti-HA and anti-FLAG monoclonal antibodies (mAbs), and ASFV-specific convalescent serum was used to validate authenticity of the expressed antigens (FIG. 2B). Auto-cleavage of the 2A peptide linker is verifiable using Western blot probed with anti-tag mAbs. Peptides were used to validate the predicted SLA I binding motifs (FIG. 3).

In some forms of the present disclosure, at least one ASFV nucleic acid sequence encoding at least one of SEQ ID NOS. 1-101 is inserted into an appropriate vector for administration to a subject. After administration to the subject, the recombinant construct expresses at least one sequence in vivo. In preferred forms, more than one ASFV nucleic acid sequence is included in a multicistronic cassette and the entire cassette is inserted into the vector for administration and in vivo expression. In some forms, the cassette includes 1, 2, 3, 4, 5, 6, 7, or more DNA sequences encoding 1, 2, 3, 4, 5, 6, 7, or more of SEQ ID NOS. 1-101. In some forms, the cassette is inserted into a conventional vector. In some forms, the cassette is an adenovirus, baculovirus, or lentivirus vector. In some forms, the vector is replication incompetent. In some forms, the vector is a single-cycle replicon adenovirus. In some forms, the vector is an attenuated bovine parainfluenza virus type 3 genotype c (BPIV3c).

The preferred methods of the present disclosure will begin with synthesis of chimeric genes encoding multiple ASFV antigens codon-optimized for protein expression in swine cells. Some preferred sequences are selected from the group consisting of nucleotide sequences that encode SEQ ID NOS. 1-101 or sequences having at least 80%, more preferably 85%, still more preferably 90%, even more preferably 91%, 92%, 93%, 94%, 95%, 96%, 97%, 98%, 99%, or 100% sequence identity or sequence homology to SEQ ID NOS. 1-101. One or more nucleotide sequences encoding one or more than one of SEQ ID NOS. 1-101 can be synthesized and cloned into a plasmid vector. The resultant construct(s) is used to generate recombinant viral expression construct(s). Protein expression by the constructs was evaluated by immunocytometric analysis using anti-HA and anti-FLAG monoclonal antibodies (mAbs), and ASFV-specific convalescent serum was used to validate authenticity of the expressed antigens (FIG. 2B). The multicistronic expression cassettes were formed using the constructs and modified to add, in-frame, a HA-tag at the N-termini and a FLAG-tag at the C-termini (FIG. 2A). The amino acid sequences of the multicistronic cassettes were used to design synthetic genes codon-optimized for protein expression in swine cells. Recombinant DNA and SC-Ad expression constructs were generated using the synthetic genes (FIG. 2B).

In some forms, the methods of the present disclosure will begin with the isolation of ASFV DNA. Any ASFV sequence can be used for purposes of the present disclosure. Some preferred sequences are selected from the group consisting of nucleotide sequences that encode SEQ ID NOS. 1-101 or sequences having at least 80%, more preferably 85%, still more preferably 90%, even more preferably 91%, 92%, 93%, 94%, 95%, 96%, 97%, 98%, 99%, or 100% sequence identity or sequence homology to SEQ ID NOS. 1-101. One or more nucleotide sequences encoding one or more than one of SEQ ID NOS. 1-101 can be isolated and cloned into the vector. In some forms, the ASFV DNA is preferably amplified using PCR methods. The resulting DNA is then cloned into the transfer vector.

In one aspect of the present disclosure, a method for generating a recombinant viral construct containing ASFV DNA is provided. This method generally comprises the steps of: 1) cloning at least one recombinant ASFV DNA sequence into a transfer vector; and 2) shuttling the portion of the transfer construct containing the recombinant ASFV sequence into a viral vector, to generate the recombinant viral construct. As noted above, some preferred ASFV nucleotide or DNA sequences encode protein sequences having at least 80%, more preferably 85%, still more preferably 90%, even more preferably 91%, 92%, 93%, 94%, 95%, 96%, 97%, 98%, 99%, or 100% sequence identity or sequence homology to SEQ ID NOS. 1-101.

According to a further aspect, the ASFV DNA can be amplified prior to step 1) in vitro. In vitro methods for amplifying the ASFV DNA and cloning in vitro amplified ASFV DNA into a transfer vector and suitable transfer vectors are described above or known to a person skilled in the art. Thus according to a further aspect, the present disclosure relates to a method for generating a recombinant viral construct containing ASFV DNA and expressing at least one desired ASFV protein comprising the steps of: 1) Synthesis of codon-optimized gene or amplifying ASFV DNA in vitro, 2) cloning the ASFV DNA into a transfer vector; and 3) shuttling a portion thereof containing the recombinant ASFV DNA into a viral vector to generate the recombinant viral construct. In some forms, the ASFV DNA sequences encode protein sequences having at least 80%, more preferably 85%, still more preferably 90%, even more preferably 91%, 92%, 93%, 94%, 95%, 96%, 97%, 98%, 99%, or 100% sequence identity or sequence homology to at least one sequence of SEQ ID NOS. 1-101. In some forms, the recombinant construct is administered for in vivo protein expression and in other forms, the protein(s) is expressed, recovered, and administered to the subject.

In another aspect of the present disclosure, a method for preparing a composition, preferably an immunogenic composition, such as a vaccine, for invoking an immune response against ASFV is provided. Generally, this method includes the steps of generating a viral expression construct, wherein the construct comprises 1) recombinant DNA from at least one DNA sequence of ASFV that encodes at least one desired antigen, 2) infecting cells in growth media with the recombinant virus, 3) causing the virus to express the recombinant protein from ASFV, 4) recovering the expressed recombinant protein, 5) and, in some forms, preparing the composition by combining the recovered protein with a suitable adjuvant and/or other pharmaceutically acceptable carrier. It is understood that adjuvants are not required to practice the methods nor to combine with the antigens of the disclosure in compositions. In some preferred forms, the composition also includes at least a portion of the viral construct expressing said ASFV protein, and/or a portion of the cell culture supernate.

In another aspect of the present disclosure, a method for preparing an immunogenic composition, such as a vaccine, for invoking an immune response against ASFV comprises the steps of 1) expressing and recovering ASFV protein, and 2) admixing the recovered protein with a suitable pharmaceutical-acceptable or veterinary-acceptable carrier. As used herein, “a pharmaceutical-acceptable carrier” or “veterinary-acceptable carrier” includes any and all solvents, dispersion media, coatings, stabilizing agents, diluents, preservatives, antibacterial and antifungal agents, isotonic agents, adsorption delaying agents, and the like. In one preferred form, the composition provided herewith, contains ASFV protein recovered from in vitro cultured cells, wherein said cells were infected with a recombinant viral construct containing ASFV DNA and expressing ASFV protein, and wherein the cell culture was treated to inactivate the viral construct, and an equivalent concentration of a neutralization agent was added, and wherein both an adjuvant and physiological saline are also added. As with the other aspects, the ASFV protein preferably has at least 80%, more preferably 85%, still more preferably 90%, even more preferably 91%, 92%, 93%, 94%, 95%, 96%, 97%, 98%, 99%, or 100% sequence identity or sequence homology to SEQ ID NOS. 1-101. When included, the amount of physiological saline is preferably about 50 to about 90% (v/v), more preferably about 60 to 80% (v/v), still more preferably about 70% (v/v). Optionally, this method can also include the addition of a protectant. A protectant as used herein, refers to an anti-microbiological active agent, such as for example Gentamycin, Merthiolate, and the like. In particular adding a protectant is most preferred for the preparation of a multi-dose composition. Those anti-microbiological active agents are added in concentrations effective to prevent the composition of interest from any microbiological contamination or for inhibition of any microbiological growth within the composition of interest.

The methods of the present disclosure can also comprise the addition of any stabilizing agent, such as for example saccharides, trehalose, mannitol, saccharose and the like, to increase and/or maintain product shelf-life and/or to enhance stability.

In another aspect of the present disclosure, products resulting from the methods as described above are provided. In particular, the present disclosure relates to a composition of matter comprising a construct having therein at least one ASFV nucleic acid sequence. In some forms, the ASFV nucleic acid sequence(s) encodes a protein having at least 80%, more preferably 85%, still more preferably 90%, even more preferably 91%, 92%, 93%, 94%, 95%, 96%, 97%, 98%, 99%, or 100% sequence identity or sequence homology to SEQ ID NOS. 1-101. In some forms, the vector is a live vector. In some forms, the vector is a replication incompetent vector, such as a recombinant adenovirus. In other forms, the multicistronic expression cassette utilizes a single cycle replicon virus, preferably a single cycle replicon adenovirus, as the vector. In still other forms, the multicistronic cassette utilizes an attenuated bovine parainfluenza virus type 3 genotype c (BPIV3c) as the vector. In some forms, the vector including the ASFV nucleic acid sequence(s) is administered to a subject as described herein. In other forms, the vector expresses the nucleic acid sequences in vitro and the resulting recombinant ASFV polypeptides or proteins are recovered and administered to a subject as described in this disclosure. Of course, it is understood that the ASFV polypeptide used in an immunogenic composition in accordance with the present disclosure can be derived in any fashion including isolation and purification, standard protein synthesis, and recombinant methodology.

In a further aspect of the present disclosure, an immunogenic composition effective for lessening the severity of clinical symptoms associated with ASFV infection comprising at least one ASFV nucleic acid is provided. Preferably, the ASFV nucleic acid encodes 1) any polypeptide that is at least 80%, more preferably 85%, still more preferably 90%, even more preferably 91%, 92%, 93%, 94%, 95%, 96%, 97%, 98%, 99%, or 100% homologous to the polypeptide of any one of SEQ ID NOS: 1-101, or any combination thereof; 2) any immunogenic portion of the polypeptides of 1); and, 3) a polypeptide equivalent to (due to the degeneracy of the genetic code) one encoded by a DNA comprising the sequence of SEQ ID NOS: 1-101. As noted above, it is particularly preferred to have multiple sequences encoding any one of SEQ ID NOS: 1-101 included in the immunogenic composition and even more preferred to have them combined together in a multicistronic expression cassette.

According to a further aspect, at least one ASFV protein is provided in the immunological composition at an antigen inclusion level effective for inducing the desired immune response, namely reducing the incidence of or lessening the severity of clinical signs resulting from ASFV infection. Preferably, the ASFV protein inclusion level is at least 25 μg antigen/ml of the final immunogenic composition (μg/ml), more preferably from about 25 to about 400 μg/ml.

The at least one nucleotide sequence, polypeptide, or polypeptide encoded by the multicistronic expression cassette is incorporated into a composition that can be administered to an animal susceptible to ASFV infection. In preferred forms, the composition may also include additional components known to those of skill in the art (see also Remington's Pharmaceutical Sciences. (1990). 18th ed. Mack Publ., Easton).

Those of skill in the art will understand that the composition herein may incorporate known injectable, physiologically acceptable, sterile solutions. For preparing a ready-to-use solution for parenteral injection or infusion, aqueous isotonic solutions, such as e.g. saline or corresponding plasma protein solutions are readily available. In addition, the immunogenic and vaccine compositions of the present disclosure can include diluents, isotonic agents, stabilizers, or adjuvants. Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylenediaminetetraacetic acid, among others. Suitable adjuvants are those described above. Oral forms of the composition are also envisioned and in some forms, preferred.

The immunogenic compositions described herein can further include one or more other immunomodulatory agents such as, e. g., interleukins, interferons, or other cytokines. The immunogenic compositions can also include Gentamicin. In another preferred embodiment, the present disclosure contemplates vaccine compositions comprising from about 1 ug/ml to about 60 μg/ml of antibiotics, and more preferably less than about 30 μg/ml of antibiotics.

It will be found that the immunogenic compositions comprising live-vectored multivalent immunogenic compositions as provided herewith are very effective in reducing the severity of or incidence of clinical signs associated with ASFV infections up to and including the prevention of such signs.

Another aspect of the present disclosure relates to a kit. Generally the kit includes a container comprising at least one dose of the live-vectored multivalent immunogenic composition of ASFV nucleotide sequences as provided in this disclosure.

In another aspect of the present disclosure, a kit is provided. Generally, the kit includes a container comprising at least one does of at least one ASFV recombinant protein as described herein. In some forms, one dose comprises at least 2 μg ASFV protein. Said container can comprise from 1 to 250 doses of the immunogenic composition. In some preferred forms, the container contains 1, 10, 25, 50, 100, 150, 200, or 250 doses of the immunogenic composition of ASFV protein. Preferably, each of the containers comprising more than one dose of the immunogenic composition of ASFV protein further comprises an anti-microbiological active agent. Those agents are for example, antibiotics including Gentamicin and the like. Thus, one aspect of the present disclosure relates to a container that comprises from 1 to 250 doses of the immunogenic composition of ASFV protein, wherein one dose comprises at least 2 μg ASFV protein, and Gentamicin, preferably from about 1 μg/ml to about 60 μg/ml of antibiotics, and more preferably less than about 30 μg/ml. In preferred forms, the kit also includes an instruction manual, including the information for the administration of at least one dose of the immunogenic composition of ASFV protein into animals, preferably pigs and piglets to lessen the incidence and/or severity of clinical symptoms associated with ASFV infection. Moreover, according to a further aspect, said instruction manual comprises the information of a second or further administration(s) of at least one dose of the immunogenic composition of ASFV, wherein the second administration or any further administration is at least 14 days beyond the initial or any former administration. In some preferred forms, said instruction manual also includes the information, to administer an immune stimulant. Preferably, said immune stimulant shall be given at least twice. Preferably, at least 14, more preferably at least 21, and even more preferably at least 28 days are between the first and the second or any further administration of the immune stimulant. Preferably, the immune stimulant is given at least 10 days, preferably 15, even more preferably 20, and still even more preferably at least 22 days beyond the initial administration of the immunogenic composition of ASFV protein. It is understood that any immune stimulant known to a person skilled in the art can also be used. “Immune stimulant” as used herein, means any agent or composition that can trigger a general immune response, preferably without initiating or increasing a specific immune response, for example the immune response against a specific pathogen. It is further instructed to administer the immune stimulant in a suitable dose. The kit may also comprise a second container, including at least one dose of the immune stimulant.

A further aspect of the present disclosure relates to the kits as described above, comprising the immunogenic composition of ASFV as provided herewith and the instruction manual, wherein the instruction manual further includes the information to administer the ASFV immunogenic composition together, or around the same time as, with an immunogenic composition that comprises an additional antigen effective for reducing the severity of or incidence of clinical signs related to another porcine pathogen. Preferably, the manual contains the information of when the ASFV-containing composition and the immunogenic composition that comprises an additional antigen are administered.

A further aspect, relates to the use of any of the compositions provided herewith as a medicament, preferably as a veterinary medicament, even more preferably as a vaccine. Moreover, the present disclosure also relates to the use of any of the compositions described herein, for the preparation of a medicament for lessening the severity of clinical symptoms associated with ASFV infection. Preferably, the medicament is for the prevention of ASFV infection in swine, even more preferably in piglets.

A further aspect relates to a method for (1) the prevention of an infection, or re-infection with ASFV or (2) the reduction in incidence or severity of or elimination of clinical symptoms caused by ASFV in a subject, comprising administering any of the immunogenic compositions provided herewith to a subject in need thereof. In some forms, the subject is a mammal, such as a pig. It is understood that the reduction is in comparison to a subject that has not received an administration of a composition of the present disclosure. Preferably, one dose or two doses of the immunogenic composition is/are administered. A further aspect relates to the method of treatment as described above, wherein a second application or administration of the immunogenic composition is administered. Preferably, the second administration is done with the same immunogenic composition, preferably having the same amount of ASFV antigen (protein or live-vectored nucleic acid). Preferably, the second administration is done at least 14 days beyond the initial administration, even more preferably at least 4 weeks beyond the initial administration. In preferred forms, the method is effective after just a single dose of the immunogenic composition and does not require a second or subsequent administration in order to confer the protective benefits upon the subject.

According to a further aspect, the present disclosure provides a multivalent combination vaccine which includes an immunological agent effective for reducing the incidence of or lessening the severity of ASFV infection, and at least one immunological active component against another disease-causing organism in swine.

In particular the immunological agent effective for reducing the incidence of or lessening the severity of ASFV infection is an ASFV antigen or protein as described herein. Preferably, said ASFV antigen encodes protein having, or is a protein that has at least 80%, more preferably 85%, still more preferably 90%, even more preferably 91%, 92%, 93%, 94%, 95%, 96%, 97%, 98%, 99%, or 100% sequence homology with any one of SEQ ID NOS: 1-101 and any combination thereof as provided herewith.

Preferably the other disease-causing organism in swine is selected from the group consisting of: Actinobacillus pleuropneumonia; Adenovirus; Alphavirus such as Eastern equine encephalomyelitis viruses; Bordetella bronchiseptica; Brachyspira spp., preferably B. hyodyentheriae; B. piosicoli, Brucella suis, preferably biovars 1, 2, and 3; Classical swine fever virus; Clostridium spp., preferably Cl. difficile, Cl. perfringens types A, B, and C, Cl. novyi, Cl. septicum, Cl. tetani; Coronavirus, preferably Porcine Respiratory Corona virus; Eperythrozoonosis suis; Erysipelothrix rhsiopathiae; Escherichia coli; Haemophilus parasuis, preferably subtypes 1, 7 and 14: Hemagglutinating encephalomyelitis virus; Japanese Encephalitis Virus; Lawsonia intracellularis; Leptospira spp.; preferably Leptospira australis; Leptospira canicola; Leptospira grippotyphosa; Leptospira icterohaemorrhagicae; and Leptospira interrogans; Leptospira pomona; Leptospira tarassovi; Mycobacterium spp. preferably M. avium; M. intracellulare; and M. bovis; Mycoplasma hyopneumoniae (M hyo); Pasteurella multocida; Porcine circovirus; Porcine cytomegalovirus; Porcine Parvovirus; Porcine Reproductive and Respiratory Syndrome (PRRS) Virus; Pseudorabies virus; Rotavirus; Salmonella spp.; preferably S. thyhimurium; and S. choleraesuis; Staph. hyicus; Staphylococcus spp. preferably Streptococcus spp., preferably Strep. suis; Swine herpes virus; Swine Influenza Virus; Swine pox virus; Swine pox virus; Vesicular stomatitis virus; Virus of vesicular exanthema of swine; Leptospira hardjo; and/or Mycoplasma hyosynoviae.

Nucleotide, polynucleotide or nucleic acid sequence will be understood according to the present disclosure as meaning both a double-stranded or single-stranded DNA in the monomeric and dimeric (so-called in tandem) forms and the transcription products of said DNAs.

It must be understood that the present disclosure does not relate to the genomic nucleotide sequences taken in their natural environment, that is to say in the natural state. It concerns sequences which it has been possible to isolate, purify or partially purify, starting from separation methods such as, for example, ion-exchange chromatography, by exclusion based on molecular size, or by affinity, or alternatively fractionation techniques based on solubility in different solvents, or starting from methods of genetic engineering such as amplification, cloning and subcloning, it being possible for the sequences of the disclosure to be carried by vectors.

Among said nucleotide sequences according to the disclosure, those coding for polypeptides, such as, for example, the sequences SEQ ID NOS. 1-101 and any combination thereof. The nucleotide sequence fragments according to the disclosure can be obtained, for example, by specific amplification, such as PCR, or after digestion with appropriate restriction enzymes of nucleotide sequences according to the disclosure, these methods in particular being described in the work of Sambrook et al., 1989. Said representative fragments can likewise be obtained by chemical synthesis when their size is not very large and according to methods well known to persons skilled in the art.

Modified nucleotide sequence will be understood as meaning any nucleotide sequence obtained by mutagenesis according to techniques well known to the person skilled in the art, and containing modifications with respect to the normal sequences according to the disclosure, for example mutations in the regulatory and/or promoter sequences of polypeptide expression, especially leading to a modification of the rate of expression of said polypeptide or to a modulation of the replicative cycle.

In the present description, the terms polypeptide, peptide and protein are interchangeable.

It must be understood that the disclosure does not relate to the polypeptides in natural form, that is to say that they are not taken in their natural environment but that they can be isolated or obtained by purification from natural sources, or else obtained by genetic recombination, or alternatively by chemical synthesis and that they can thus contain unnatural amino acids.

Polypeptide fragment according to the disclosure is understood as designating a polypeptide containing at least 5 consecutive amino acids, preferably 10 consecutive amino acids or 15 consecutive amino acids.

The nucleotide sequences coding for a polypeptide according to the disclosure are likewise part of the disclosure.

The disclosure likewise relates to nucleotide sequences utilizable as a primer or probe, characterized in that said sequences are selected from the nucleotide sequences according to the disclosure.

Another subject of the present disclosure is a vector for the cloning and/or expression of a sequence, characterized in that it contains a nucleotide sequence according to the disclosure.

The vectors according to the disclosure, characterized in that they contain the elements allowing the expression and/or the secretion of said nucleotide sequences in a determined host cell, are likewise part of the disclosure. The vector must then contain a promoter, signals of initiation and termination of translation, as well as appropriate regions of regulation of transcription. It must be able to be maintained stably in the host cell and can optionally have particular signals specifying the secretion of the translated protein. These different elements are chosen as a function of the host cell used. To this end, the nucleotide sequences according to the disclosure can be inserted into autonomous replication vectors within the chosen host, or integrated vectors of the chosen host. Such vectors will be prepared according to the methods currently used by the person skilled in the art, and it will be possible to introduce the clones resulting therefrom into an appropriate host by standard methods, such as, for example, oral administration, injection, lipofection, transdermal administration, electroporation and thermal shock. The vectors according to the disclosure are, for example, vectors of plasmid or viral origin. Preferred vectors for the expression of polypeptides of the disclosure include a single-cycle replicon adenovirus or an attenuated bovine parainfluenza virus type 3 genotype 3C (BPIV3C).

Advantageously, the antigenic determinant is such that it is capable of inducing a humoral and/or cellular response. It will be possible for such a determinant to comprise a polypeptide according to the disclosure in glycosylated form used with a view to obtaining immunogenic compositions capable of inducing the synthesis of antibodies directed against multiple epitopes. Said polypeptides or their glycosylated fragments are likewise part of the disclosure. These hybrid molecules can be formed, in part, of a polypeptide carrier molecule or of fragments thereof according to the disclosure, associated with a possibly immunogenic part, in particular an epitope of the diphtheria toxin, the tetanus toxin, a surface antigen of the hepatitis B virus (patent FR 79 21811), the VP1 antigen of the poliomyelitis virus or any other viral or bacterial toxin or antigen. The procedures for synthesis of hybrid molecules encompass the methods used in genetic engineering for constructing hybrid nucleotide sequences coding for the polypeptide sequences sought. It will be possible, for example, to refer advantageously to the technique for obtainment of genes coding for fusion proteins described by Minton in 1984. Said hybrid nucleotide sequences coding for a hybrid polypeptide as well as the hybrid polypeptides according to the disclosure characterized in that they are recombinant polypeptides obtained by the expression of said hybrid nucleotide sequences are likewise part of the disclosure.

The disclosure likewise comprises the vectors characterized in that they contain one of said hybrid nucleotide sequences. The host cells transformed by said vectors, the transgenic animals comprising one of said transformed cells as well as the procedures for preparation of recombinant polypeptides using said vectors, said transformed cells and/or said transgenic animals are, of course, likewise part of the disclosure.

The disclosure likewise relates to a pharmaceutical composition comprising a compound selected from the following compounds: a) a nucleotide sequence according to the disclosure; b) a polypeptide according to the disclosure; c) a vector, a viral particle or a cell transformed according to the disclosure; d) an antibody according to the disclosure; and e) a compound capable of being selected by a selection method according to the disclosure; possibly in combination with a pharmaceutically acceptable carrier and, if need be, with one or more adjuvants of the appropriate immunity.

The disclosure also relates to an immunogenic and/or vaccine composition, characterized in that it comprises a compound selected from the following compounds: a) a nucleotide sequence according to the disclosure; b) a polypeptide according to the disclosure; c) a vector or a viral particle according to the disclosure; and d) a cell according to the disclosure.

In one embodiment, the vaccine composition according to the disclosure is characterized in that it comprises a mixture of at least two of said compounds a), b), c) and d) above and in that one of the two said compounds is related to the ASFV.

In another embodiment of the disclosure, the vaccine composition is characterized in that it comprises at least one compound a), b), c), or d) above which is related to ASFV.

A compound related to the ASFV is understood here as respectively designating a compound obtained from the genomic sequence of the ASFV. Preferably the compound will be selected from the group consisting of at least one nucleic acid sequence encoding for a sequence having at least 80%, more preferably 85%, still more preferably 90%, even more preferably 91%, 92%, 93%, 94%, 95%, 96%, 97%, 98%, 99%, or 100% sequence homology with any one or more of SEQ ID NOS: 1-101, and any combination thereof, a polypeptide sequence having at least 80%, more preferably 85%, still more preferably 90%, even more preferably 91%, 92%, 93%, 94%, 95%, 96%, 97%, 98%, 99%, or 100% sequence homology with any one or more of SEQ ID NOS: 1-101, and any combination thereof, and any combination of at least one nucleic acid sequence and at least one polypeptide sequence, as described herein.

The disclosure is additionally aimed at an immunogenic and/or vaccine composition, characterized in that it comprises at least one of the following compounds: 1) a nucleotide sequence encoding any one of SEQ ID NOS. 1-101 or one of their fragments or homologues; 2) a polypeptide selected from the group consisting of SEQ ID NOS. 1-101; 3) a vector or a viral particle comprising a nucleotide sequence of 1); 4) a transformed cell capable of expressing a polypeptide of 2); or 5) a mixture of at least two of said compounds.

The disclosure also comprises an immunogenic and/or vaccine composition according to the disclosure, characterized in that it comprises said mixture of at least two of said compounds as a combination product for simultaneous, separate or protracted use for the prevention or the treatment of infection by ASFV.

The disclosure is likewise directed at a pharmaceutical composition according to the disclosure, for the prevention or the treatment of an infection by ASFV.

It is understood that “prevention” as used in the present disclosure, includes the complete prevention of infection by ASFV, but also encompasses a reduction in the severity of or incidence of clinical signs associated with or caused by ASFV infection. Such prevention is also referred to herein as a protective effect.

The disclosure likewise concerns the use of a composition according to the disclosure, for the preparation of a medicament intended for the prevention or the treatment of infection by ASFV.

The live-vectored multivalent immunogenic compositions as described herein and the polypeptides of the disclosure entering into the immunogenic or vaccine compositions according to the disclosure can be selected by techniques known to the person skilled in the art such as, for example, depending on the capacity of said polypeptides to stimulate the T cells, which is translated, for example, by their proliferation or the secretion of interleukins, and which leads to the production of antibodies directed against said polypeptides.

In pigs, as in mice, in which a weight dose of the vaccine composition comparable to the dose used in man is administered, the antibody reaction is tested by taking of the serum followed by a study of the formation of a complex between the antibodies present in the serum and the antigen of the vaccine composition, according to the usual techniques.

The pharmaceutical compositions according to the disclosure will contain an effective quantity of the compounds of the disclosure, that is to say in sufficient quantity of said compound(s) allowing the desired effect to be obtained, such as, for example, the modulation of the cellular replication of ASFV. The person skilled in the art will know how to determine this quantity, as a function, for example, of the age and of the weight of the individual to be treated, of the state of advancement of the pathology, of the possible secondary effects and by means of a test of evaluation of the effects obtained on a population range, these tests being known in these fields of application.

According to the disclosure, said vaccine combinations will preferably be combined with a pharmaceutically or veterinary acceptable carrier and, if need be, with one or more adjuvants of the appropriate immunity.

According to another embodiment of the vaccine composition according to the disclosure, the nucleotide sequence, preferably a DNA, is complexed with DEAE-dextran (Pagano et al., 1967) or with nuclear proteins (Kaneda et al., 1989), with lipids (Felgner et al., 1987) or encapsulated in liposomes (Fraley et al., 1980) or else introduced in the form of a gel facilitating its transfection into the cells (Midoux et al., 1993, Pastore et al., 1994). The polynucleotide or the vector according to the disclosure can also be in suspension in a buffer solution or be combined with liposomes.

These compounds can be administered by the systemic route, in particular by the intravenous route, by the intramuscular, intradermal or subcutaneous route, or by the oral route. In a more preferred manner, the vaccine composition comprising polypeptides according to the disclosure will be administered by the intramuscular route, through the food or by nebulization only once, or several times, staggered over time.

Their administration modes, dosages and optimum pharmaceutical forms can be determined according to the criteria generally taken into account in the establishment of a treatment adapted to an animal such as, for example, the age or the weight, the seriousness of its general condition, the tolerance to the treatment and the secondary effects noted. Preferably, the vaccine of the present disclosure is administered in an amount that is protective or provides a protective effect against ASFV infection. In some forms, the composition of the disclosure is administered

An immunologically effective amount of the vaccines or immunogenic compositions of the present disclosure is administered to a pig in need of protection against clinical signs of ASFV infection. The immunologically effective amount or the immunogenic amount that inoculates the pig can be easily determined or readily titrated by routine testing. An effective amount is one in which a sufficient immunological response to the vaccine is attained to protect the pig exposed to the virus which causes ASFV signs. Preferably, the pig is protected to an extent in which one to all of the adverse physiological symptoms or effects of the viral disease are significantly reduced, ameliorated or totally prevented.

The vaccine can be administered in a single dose or in repeated doses with single doses being preferred. Single dose vaccines provide protection after a single dose without the need for any booster or subsequent dosages. Protection can include the complete prevention of clinical signs of infection, or a lessening of the severity, duration, or likelihood of the manifestation of one or more clinical signs of infection.

Desirably, the vaccine is administered to a pig not yet exposed to the ASFV virus.

When administered as a liquid, the present vaccine may be prepared in the form of an aqueous solution, syrup, an elixir, a tincture and the like. Such formulations are known in the art and are typically prepared by dissolution of the antigen and other typical additives in the appropriate carrier or solvent systems. Suitable carriers or solvents include, but are not limited to, water, saline, ethanol, ethylene glycol, glycerol, etc. Typical additives are, for example, certified dyes, flavors, sweeteners and antimicrobial preservatives such as thimerosal (sodium ethylmercurithiosalicylate). Such solutions may be stabilized, for example, by addition of partially hydrolyzed gelatin, sorbitol or cell culture medium, and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Liquid formulations also may include suspensions and emulsions that contain suspending or emulsifying agents in combination with other standard co-formulants. These types of liquid formulations may be prepared by conventional methods. Suspensions, for example, may be prepared using a colloid mill. Emulsions, for example, may be prepared using a homogenizer.

Parenteral formulations, designed for injection into body fluid systems, require proper isotonicity and pH buffering to the corresponding levels of porcine body fluids. Isotonicity can be appropriately adjusted with sodium chloride and other salts as needed. Suitable solvents, such as ethanol or propylene glycol, can be used to increase the solubility of the ingredients in the formulation and the stability of the liquid preparation. Further additives that can be employed in the present vaccine include, but are not limited to, dextrose, conventional antioxidants and conventional chelating agents such as ethylenediamine tetraacetic acid (EDTA). Parenteral dosage forms must also be sterilized prior to use.

The following examples demonstrate certain aspects of the present disclosure. However, it is to be understood that these examples are for illustration only and do not purport to be wholly definitive as to conditions and scope of this disclosure. It should be appreciated that when typical reaction conditions (e.g., temperature, reaction times, etc.) have been given, the conditions both above and below the specified ranges can also be used, though generally less conveniently. The examples are conducted at room temperature (about 23° C. to about 28° C.) and at atmospheric pressure. All parts and percentages referred to herein are on a weight basis and all temperatures are expressed in degrees centigrade unless otherwise specified. Further unless noted otherwise, all components of the disclosure are understood to be disclosed to cover “comprising”, “consisting essentially of”, and “consisting of” claim language as those terms are commonly used in patent claims.

Another aspect of the present disclosure is the preparation of the combination vaccine(s) or immunogenic compositions. Such combinations can be between the different vaccine components described herein. For example, a vaccine of the present disclosure can include both protein portions and DNA portions of ASFV, as described herein, which are administered concurrently or separately. Additionally, the combinations can be between the ASFV vaccine components described herein and antigens of other disease-causing organisms, such as those described above.

According to the present disclosure, an effective amount of a combination vaccine administered to pigs provides effective immunity or a protective effect against microbiological infections caused by ASFV and at least one further pathogen. Preferred combinations of antigens for the treatment and prophylaxis of microbiological diseases in pigs are listed above.

According to a further embodiment, the combination vaccine is administered to pigs in one or two doses at an interval of about 2 to 4 weeks. For example, the first administration is performed when the animal is about 2 to 3 weeks to about 8 weeks of age. The second administration is performed about 1 to about 4 weeks after the first administration of the first vaccination. According to a further embodiment, revaccination is performed in an interval of 3 to 12 month after administration of the second dose. Administration of subsequent vaccine doses is preferably done on a 6 month to an annual basis. In another preferred embodiment, animals vaccinated before the age of about 2 to 3 weeks should be revaccinated. Administration of subsequent vaccine doses is preferably done on an annual basis. In the event that one of the components of the combination vaccine is effective after just a single dose, such component needs to only be administered a single time with the other component(s) administered according to their preferred regimen.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A is table illustrating the results from ASFV ORF down selection criteria;

FIG. 1B is flow diagram illustrating the ASFV ORF down selection criteria;

FIG. 2A is a flow diagram illustrating the design of multicistronic cassettes;

FIG. 2 B is a set of photographs illustrating the validation of protein expression;

FIG. 3 is a graph illustrating IFN-γ response to predicted SLA I binding motifs;

FIG. 4A is graph illustrating RD-Ad primed antibody responses against ASFV antigens p32, p54, pp62, and p′72;

FIG. 4B is a set of photographs illustrating recognition of ASF virus (Georgia 2007/1) by sera from vaccinees was validated by IFA using ASFV-infected primary porcine macrophages probed with sera from blood collected 1 week post-boost;

FIG. 5A is a graph illustrating RD-Ad primed IFN-γ responses against ASFV antigen p37 (part of pp220);

FIG. 5B is a graph illustrating RD-Ad primed IFN-γ responses against ASFV antigen p150 antigen (also part of pp220);

FIG. 6A is a graph illustrating RD-Ad primed CTL responses to p32, p54, p62, p′72, and FMD [negative control] with the ENABL adjuvant;

FIG. 6B is a graph illustrating RD-Ad primed CTL responses to p32, p54, p62, p′72, and FMD using the TXO adjuvant;

FIG. 7A is a graph illustrating survival post-challenge;

FIG. 7B is a graph illustrating viremia post-challenge;

FIG. 8A is a dot-blot analysis illustrating protein concentration of 1) p72; 2) p62; 3) p54; 4) p32; and 5) KP1712 chimera at 1:5,000 serum dilution;

FIG. 8B is a dot-blot analysis illustrating signal detection sensitivity of 4 ng p72, p54, p32, and KP1712 chimera proteins at 1:5,000 serum dilution; and

FIG. 8C is a dot-blot analysis [using 4 ng protein concentration] illustrating that only KP1712, but not p32, p54, and p72 were detectable at 1:20,000.

### DETAILED DESCRIPTION

The following detailed description and examples set forth preferred materials and procedures used in accordance with the present disclosure. It is to be understood, however, that this description and these examples are provided by way of illustration only, and nothing therein shall be deemed to be a limitation upon the overall scope of the present disclosure.

“A”, “an”, and “the” include the singular and plural forms thereof unless the context clearly indicates otherwise.

“Comprising”, “comprises”, “comprise”, “including”, “includes”, “include”, “having”, “has”, and “with” are all defined as being inclusive of the specified components as well as other unspecified components and can be used interchangeably.

An “immunogenic or immunological composition” refers to a composition of matter that comprises at least one antigen which elicits an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or γδ T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction in the severity or prevalence of, up to and including a lack of symptoms normally displayed by an infected host, a quicker recovery time and/or a lowered viral titer in the infected host.

The term “transfected into a viral vector” means, and is used as a synonym for “introducing” or “cloning” a heterologous DNA sequence encoding a desired antigen into a viral vector, such as for example into a single-cycle replicon adenovirus, an attenuated bovine parainfluenza virus type 3 genotype c (BPIV3c), or a conventional vector such as a baculovirus vector. A “transfer vector” means a DNA molecule, that includes at least one origin of replication, the heterologous ASFV DNA sequence that encodes a desired antigen, in the present case of ASFV, DNA sequences which allow the cloning of said heterologous ASFV DNA sequence into the viral vector will be included. Preferably the sequences which allow cloning of the heterologous DNA sequence into the viral vector are flanking the heterologous DNA. Even more preferably, those flanking sequences are at least homologous in parts with sequences of the viral vector. The sequence homology then allows recombination of both molecules, the viral vector, and the transfer vector to generate a recombinant viral construct containing the heterologous DNA sequence encoding a desired antigen.

“Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalene or squalene oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.). John Wiley and Sons, NY, pp 51-94 (1995) and Todd et al., Vaccine 15:564-570 (1997).

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with an allyl sucrose or with allyl pentaerythritol. Among then, there may be mentioned Carbopol 974P, 934P and 971P. Among the copolymers of maleic anhydride and alkenyl derivative, the copolymers EMA (Monsanto) which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIM adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide among many others.

Preferably, the adjuvant is added in an amount of about 100 μg to about 10 mg per dose. Even more preferably, the adjuvant is added in an amount of about 100 μg to about 10 mg per dose. Even more preferably, the adjuvant is added in an amount of about 500 μg to about 5 mg per dose. Even more preferably, the adjuvant is added in an amount of about 750 μg to about 2.5 mg per dose. Most preferably, the adjuvant is added in an amount of about 1 mg per dose.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxyl terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homologous sequence comprises at least a stretch of 50, even more preferably 100, even more preferably 250, even more preferably 500 nucleotides.

A “conservative substitution” refers to the substitution of an amino acid residue or nucleotide with another amino acid residue or nucleotide having similar characteristics or properties including size, hydrophobicity, etc., such that the overall functionality does not change significantly.

Isolated” means altered “by the hand of man” from its natural state, i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein.

“Modified polypeptide” of a polypeptide according to the disclosure is understood as designating a polypeptide obtained by genetic recombination or by chemical synthesis as will be described below, having at least one modification with respect to the normal sequence. These modifications will especially be able to bear an amino acids at the origin of a specificity, of pathogenicity and/or of virulence, or at the origin of the structural conformation, and of the capacity of membrane insertion of the polypeptide according to the disclosure. It will thus be possible to create polypeptides of equivalent, increased or decreased activity, and of equivalent, narrower, or wider specificity. Among the modified polypeptides, it is necessary to mention the polypeptides in which up to 5 amino acids can be modified, truncated at the N- or C-terminal end, or even deleted or added.

As is indicated, the modifications of the polypeptide will especially have as objective: to render it capable of modulating, of inhibiting or of inducing the expression of ASFV gene and/or capable of modulating the replication cycle of ASFV in the cell and/or the host organism, of allowing its incorporation into vaccine compositions, and/or of modifying its bioavailability as a compound for therapeutic use.

The methods allowing said modulations on eukaryotic or prokaryotic cells to be demonstrated are well known to the person skilled in the art. It is likewise well understood that it will be possible to use the nucleotide sequences coding for said modified polypeptides for said modulations, for example through vectors according to the disclosure and described below, in order, for example, to prevent or to treat the pathologies linked to the infection.

The preceding modified polypeptides can be obtained by using combinatorial chemistry, in which it is possible to systematically vary parts of the polypeptide before testing them on models, cell cultures or microorganisms for example, to select the compounds which are most active or have the properties sought.

Chemical synthesis likewise has the advantage of being able to use unnatural amino acids, or nonpeptide bonds. Thus, in order to improve the duration of life of the polypeptides according to the disclosure, it may be of interest to use unnatural amino acids, for example in D form, or else amino acid analogs, especially sulfur-containing forms, for example.

Finally; it will be possible to integrate the structure of the polypeptides according to the disclosure, its specific or modified homologous forms, into chemical structures of polypeptide type or others. Thus, it may be of interest to provide at the N- and C-terminal ends compounds not recognized by the proteases.

### EXAMPLES

The following examples illustrate embodiments of the disclosure. Nothing in these examples should be limiting to the disclosure as these are representative in nature.

### Example 1

This example ranked ASFV ORFs for their ability to bind to SLA alleles.

### Materials and Methods

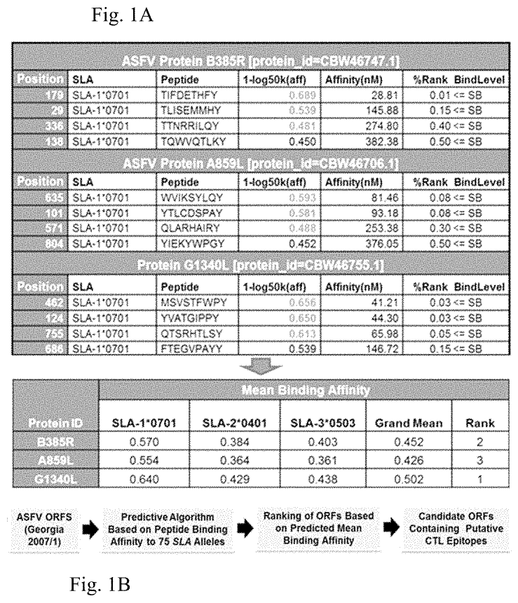

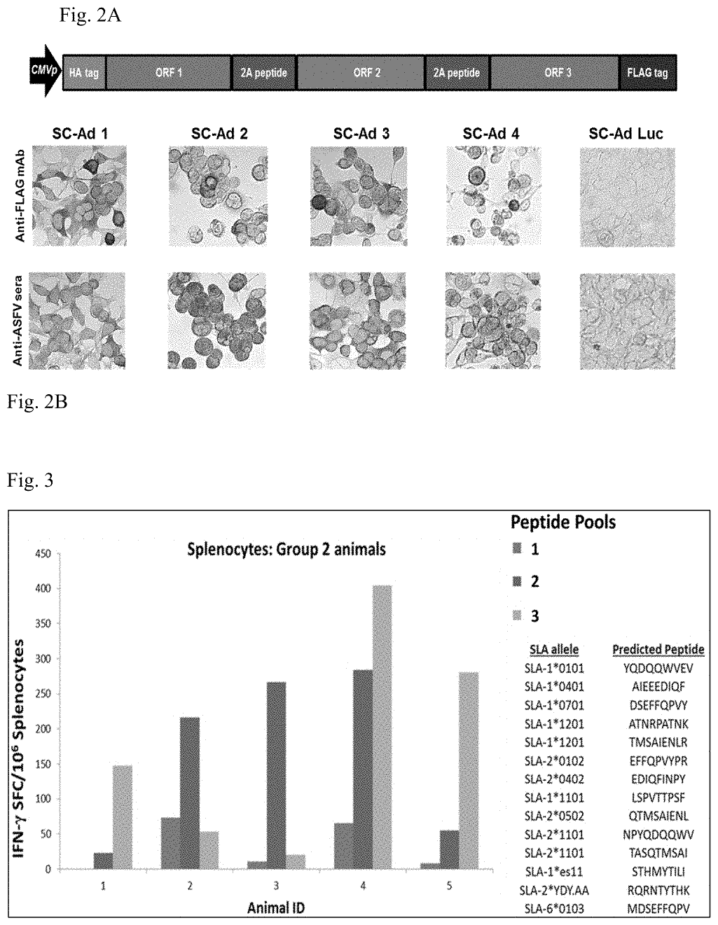

All the conserved ASFV (Georgia 2007/1) open-reading frames (ORFs) were screened for the presence of peptide motifs that bind strongly to 75 defined SLA I alleles and identified novel targets containing putative CTL epitopes (found on the web at cbs.dtu.dk/services/NetMHCpan/). The targets were ranked based on the number of putative CTL epitopes and binding affinity. As shown in FIG. 1A, ORFs were screened using 75 SLA alleles. The binding affinities of top 3 strong binders [SBs] (example in blue color) were used to calculate mean binding affinity for each ORF. The mean binding affinities from the 75 alleles were used to calculate grand mean binding affinities and the outcome was used to rank the ORFs as illustrated using means from 3 alleles. This ranking criterion is reasonable since it is based on putative epitopes as predicted by their ability to bind to SLA alleles. Three ORFs (B385R, A859L, G1340L) and three SLA alleles (SLA-1*0701; SLA-2*0401; and SLA-3*0503) were used to illustrate the down selection criteria. The algorithm, as described in NetMHCpan-4.0: Improved Peptide-MHC Class I Interaction Predictions Integrating Eluted Ligand and Peptide Binding Affinity Data. Vanessa Jurtz, Sinu Paul, Massimo Andreatta, Paolo Marcatili, Bjoern Peters and Morten Nielsen. The Journal of Immunology (2017) ji1700893; DOI: 10.4049/jimmunol.1700893; or NetMHCpan-3.0: improved prediction of binding to MHC class I molecules integrating information from multiple receptor and peptide length data sets. Morten Nielsen and Massimo Andreatta. Genome Medicine (2016): 8:33; and NetMHCpan, a method for MHC class I binding prediction beyond humans. Ilka Hoof, Bjoern Peters, John Sidney, Lasse Eggers Pedersen, Ole Lund, Soren Buus, and Morten Nielsen. Immunogenetics 61.1 (2009): 1-13] (the teachings and contents of which are incorporated by reference herein) predicts binding of peptides to known MHC molecules using Artificial Neural Networks. All the ASFV open reading frames [ORFs] selected were subjected to this process and shown to contain multiple putative strong MHC I-binding peptide motifs. MHC-1 molecules have a strong preference for binding 9-mer peptides. The output is based on predicted binding affinity and is presented as either strong binder (SB) or weak binder (WB). Some ORFs have several predicted SBs, some have one or two, whereas others have none. However, only peptides with high affinity can compete for binding to the cognate MHC molecule(s). The grand mean affinity is expected to be a representative estimated binding index for each ORF. FIG. 1B illustrates a flow diagram summarizing the down selection work flow. The targets were validated using ASFV convalescent serum followed by in vitro confirmation of the SLA class I-binding motifs using peptides. One hundred ORFs were selected (see list below) and used to design multicistronic expression cassettes separated by an efficient self-cleaving 2A peptide linker (FIG. 2A). The multicistronic expression cassettes were modified to add, in-frame, a HA-tag at the N-termini and a FLAG-tag at the C-termini (FIG. 2A). The amino acid sequences of the multicistronic cassettes were used to design synthetic genes codon-optimized for protein expression in swine cells. Recombinant DNA and SC-Ad expression constructs were generated using the synthetic genes (FIG. 2B). Protein expression by the constructs was evaluated by immunocytometric analysis using anti-HA and anti-FLAG monoclonal antibodies (mAbs), and ASFV-specific convalescent serum was used to validate authenticity of the expressed antigens (FIG. 2B). Protein expression by recombinant SC-Ad was confirmed by immunocytometric analysis of infected cells using anti-FLAG mAb (upper panel) and authenticity was validated by the ASFV convalescent serum (lower panel). Note: SC-Ad 2-4 on upper panel—most of the infected cells lifted off Virus dose was reduced for the lower panel to solve this problem. Data shown is for four constructs. Auto-cleavage of the 2A peptide linker is verifiable using Western blot probed with anti-tag mAbs. Peptides (9-mer) were used to validate the predicted ASFV (Georgia 2007/1) SLA I binding motifs by IFN-γ EliSpot assay using splenocytes from immunized pigs (FIG. 3).

### Results and Discussion

Since peptide presentation by Swine Leukocyte Antigen I (SLA I) is required to prime and expand CTLs, a strategy that utilizes an algorithm based on SLA I-peptide binding is expected to identify putative candidate antigens based on the presence of peptide motifs predicted to bind with high affinity to defined SLA I molecules.

The flow diagram in FIG. 2A illustrates the design of multicistronic cassettes for the ASFV antigens. Up to seven ORFs were included in a single cassette since the adenovirus vector can accommodate insert size of up to 8 kb. Codon-optimized synthetic genes were first cloned into pCDNA3 mammalian expression vector which has a strong CMV promoter (CMVp) and anti-HA/FLAG mAbs were used to confirm protein expression (data not shown). The photographs in FIG. 2B illustrate the validation of protein expression. The graph in FIG. 3 illustrates IFN-γ response to predicted SLA I binding motifs. Sample data from 3 pools (20 peptides/pool) is shown. Some pigs (e.g. #4) had strong responses against multiple pools.

### Example 2

This example demonstrates RD-Ad primed antibody responses against ASFV antigens, recognition of ASF virus (Georgia 2007/1) by sera from vaccinees, RD-Ad primed IFN-γ responses against ASFV antigens, and RD-Ad primed CTL responses.

### Materials and Methods

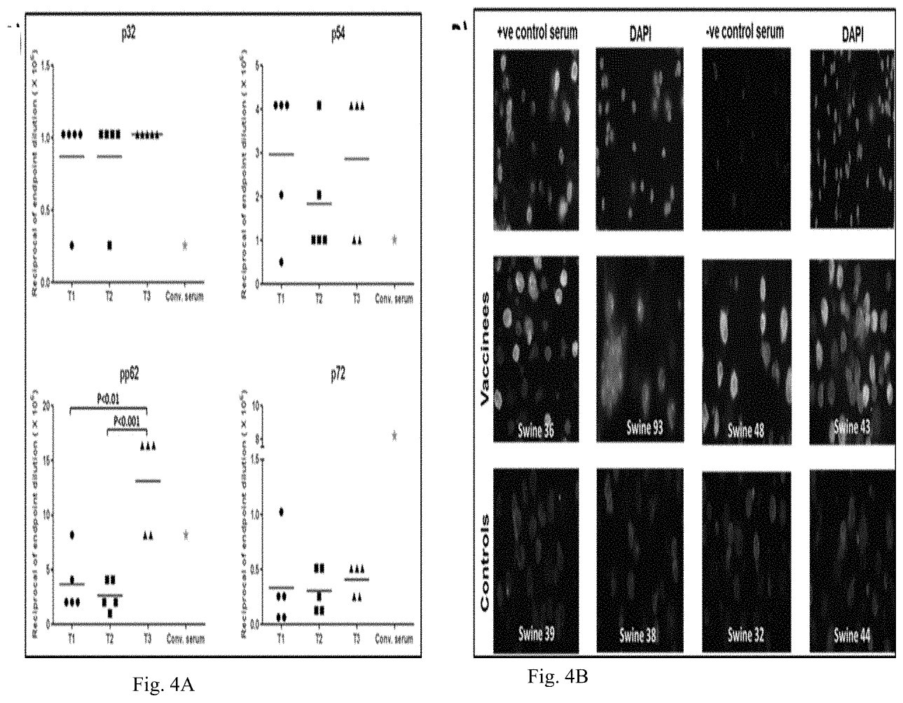

To demonstrate RD-Ad primed antibody responses against ASFV antigens, three immunogen formulations were tested. Formulation 1 (pig #s 1-5: 1×10¹⁰ ifu/adjuvant 1); Formulation 2 (pig #s 6-10: 1×10¹¹ ifu/adjuvant 1); Negative controls (pig #s 11-15: 1×10¹¹ ifu); and Formulation 3 (pig #s 16-20: 1×10¹¹ ifu/adjuvant 2). The positive control was ASFV-specific convalescent serum (red star symbol). Antibody endpoint titers were determined by ELISA (data for representative antigens is shown). Results are provided in FIG. 4A.

To demonstrate and validate recognition of ASF virus (Georgia 2007/1) by sera from vaccinees, IFA was conducted using ASFV-infected primary porcine macrophages probed with sera from blood collected 1 week post-boost. Representative data for the vaccinees and negative controls are shown in FIG. 4B.

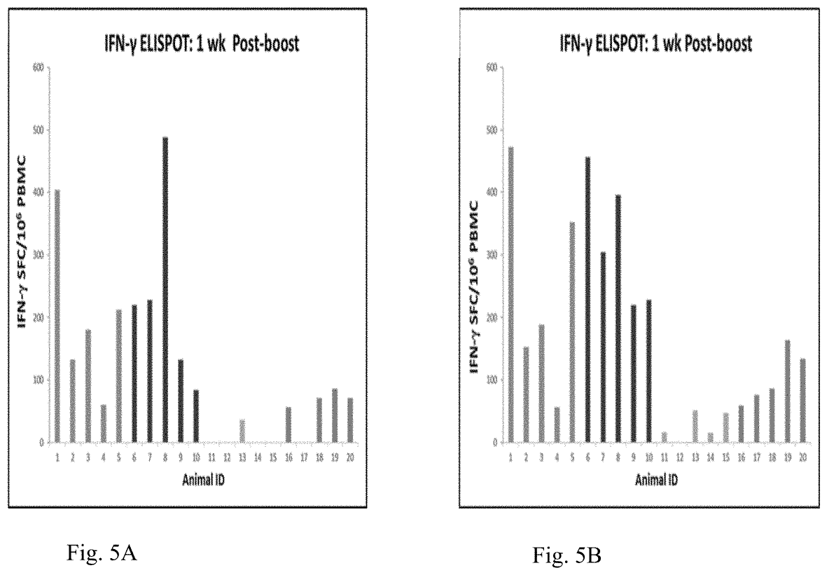

To demonstrate RD-Ad primed IFN-γ responses against ASFV antigens. IFN-γ responses for two representative antigens, p37 and p150, are shown in FIG. 5A and FIG. 5B, respectively. Both of these ASFV antigens are part of the pp220 polyprotein. Treatment groups are as indicated in FIG. 5

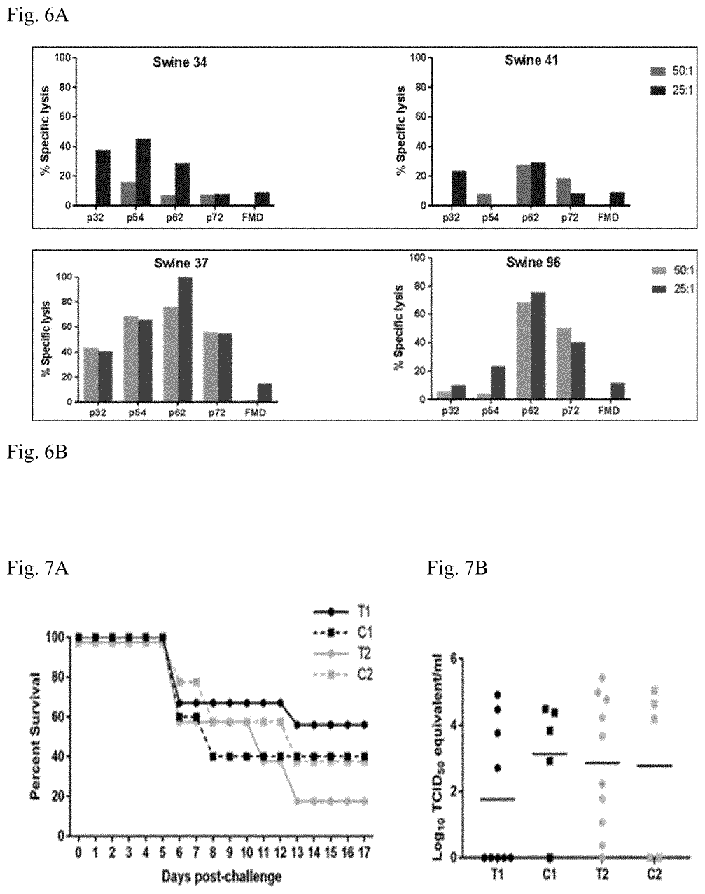

To demonstrate RD-Ad primed CTL responses, ASFV antigen-specific CTL responses were evaluated by standard ⁵¹Cr release assays using PBMCs isolated post-boost. CTL activity, at defined effector-target ratios, was determined using ⁵¹Chromium-labelled autologous skin fibroblasts (targets) transfected with DNA construct expressing cognate antigen. Fibroblasts transfected with a construct expressing an FMD antigen served as negative control targets. Priming of CTLs was influenced by the type of adjuvant used. FIG. 6A illustrates the RD-Ad ASFV antigen cocktail formulated with ENABL adjuvant (Benchmark Biolabs). FIG. 6B illustrates the RD-Ad ASFV antigen cocktail formulated with TXO adjuvant (Zoetis).

### Example 3

This example determines survival and viremia post-challenge with ASFV.

### Materials and Methods

Pigs were immunized with either RD-Ad ASFV antigen cocktail formulated in TXO adjuvant (T1) or ENABL adjuvant (T2). Negative controls received RD-Ad expressing Luciferase formulated in TXO (C1) or ENABL (C2), respectively. The pigs were challenged with 3 log₁₀ TCID₅₀ ASFV [Georgia 2007/1] administered intranasally. Viremia in blood at termination was determined by qPCR.

### Results

Results are provided in FIGS. 7A and 7B. As can be seen, 5/9 pigs from the TI group were alive and healthy 17 days post-challenge compared to 2/10 from the T2 group, 2/5 pigs from the CI and C2. However, the pigs from the T2, C1, and C2 were moribund [based on clinical scores and evaluation by attending veterinarian] and needed to be euthanized.

### Example 4

This example estimates protein concentration.

### Materials and Methods

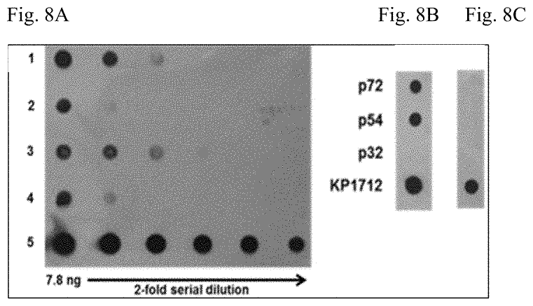

Protein concentration was estimated using BCA and antigen loads were normalized by SDS-PAGE analysis. Dot blots were probed with convalescent serum. Antigens were titrated and the blot was probed at 1:5,000 serum dilution: 1) p′72; 2) p62; 3) p54; 4) p32; and 5) KP1712 chimera. Results are provided in FIG. 8A. Probing a blot loaded with 4 ng of each antigen showed that PK1712 was better than p32, p54 & p72 at 1:5,000. Results are provided in FIG. 8B. Additionally, KP1712 was the only one detectable at 1:50,000. Notably, p32, p54, and p72 were not detectable at 1:20,000, but KP1712 was still detectable at 1:100,000 serum dilution (data not shown). Results are provided in FIG. 8C.

### Results and Discussion

It is desirable to develop a vaccine and a compatible diagnostic tool capable of differentiating infected from vaccinated animals [DIVA]. In the course of validating ASFV antigens using the ASFV convalescent serum, a chimeric antigen designated KP1712, was recognized more strongly than p32, p54, p′72, and pp62 which have previously been evaluated as diagnostic antigens. The convalescent serum is a valuable probe since it was produced by sequential infection of a pig with a series of tissue culture-adapted and wild-type viruses from genotypes I (DR11, Haiti 81, Lisbon 60, Malawi 83, and UG-61), VIII, and X (13). The serum was gamma-irradiated and provided by DHS S&T. There is a p72-based Lateral Flow Device (LFD) in the market (Ingenasa, Spain) and the OIE's ASFV reference ELISA (INgezim PPA COMPAC) utilizes virus lysate.

The results indicate that a KP1712-based LFD will likely be a superior diagnostic tool. Notably, this was confirmed by comparing the reactivity of the convalescent serum against normalized amounts of affinity purified antigens (quality control validated by Western Blot) using Dot Blot assay (FIG. 8). The sensitivity limit for the recombinant KP1712 chimera was determined to be <0.24 ng, which is 16 fold lower than for p72 (3.9 ng) and 32 fold lower than p32 and p54 (7.8 ng) (FIG. 8). Therefore, the KP1712 antigen is being used to develop a prototype Lateral Flow Device (LFD) as a highly sensitive tool for ASFV diagnosis using saliva and sera from pigs and wild boars.

Relevant to development of a subunit vaccine and a compatible DIVA diagnostic tool, the p72 antigen contains antibody and CTL epitopes. In addition, it contains multiple predicted strong SLA I binding motifs. However, the KP1712 antigen has few and weak predicted SLA I binding motifs whose affinities are below cutoff threshold. Consequently, it is rational to retain the p72 antigen in a vaccine and utilize the KP1712 as a diagnostic antigen.

### Example 5

This example prepares representative multicistronic cassettes useful for administration to subjects in need thereof.

### Materials and Methods

The selected ASFV [Georgia 2007/1] polypeptide sequences were used to design multicistronic expression cassettes separated by an efficient self-cleaving 2A peptide linker as illustrated in FIG. 2A using ORF1 [generally genes encoding large antigens: ˜3.5 kb], ORF2 [genes encoding medium size antigens: ˜2 kb], and ORF3 [genes encoding small antigens: ˜1 kb] as an example. The multicistronic cassettes contains 3-7 ORFs and overall chimeric gene size was limited to about 4.5 kb for stability and protein expression efficiency. Each multicistronic polypeptide sequence cassette was modified to add, in-frame, a HA-tag at the N-termini and a FLAG-tag at the C-termini (FIG. 2A). The resultant amino acid sequences of the multicistronic cassettes were used to design synthetic genes codon-optimized for protein expression in swine cells. The synthetic genes were cloned into pCDNA3.1+ mammalian expression vector [outsourced from GenScript], which utilizes CMV promoter to drive protein expression. SC-Ad expression constructs were generated using the synthetic genes (FIG. 2B). Protein expression by the expression constructs was evaluated by immunocytometric analysis using anti-HA and anti-FLAG monoclonal antibodies (mAbs), and ASFV-specific convalescent serum was used to validate authenticity of the expressed antigens (FIG. 2B).

### Results

The following multicistronic cassettes were generated:

### Polypeptide Sequence Used to Generate Multicistronic Expression Cassettes:

1. p220.1
(SEQ ID NO. 102)
MGNRGSSTSSRPLPSSEANIYAKLQDHIQRQTRPFSGGGYFNGGGDKNPVQHIKDYHIDSVSSKA
KLRIIEGIIRAIAKIGFKVDTKQPIEDILKDIKKQLPDPRAGSTFVKNAEKQETVCKMIADAINQEFI
DLGQDKLIDTTEGAASICRQIVLYINSLTHGLRAEYLDVHGSIENTLENIKLLNDAIKQLHERMVT
EVTKAAPNEEVINAVTMIEAVYRRLLNEQNLQINILTNFIDNILTPTQKELDKLQTDEVDIIKLLND
TNSVLGTKNFGKVLSYTLCNLGIAASVANKINKALQKVGLKVEQYLQSKNWAEFDKELDLKRF
SGLVSAENIAEFEKAVNLLRQTFNERHKILENSCAKKGGDEEKTPLDRRIEAQRLDRKHILMEFL
NKSTQAYNDFLENVKKIGIKLVKEIALTPNITRLRDALSRINDMGTIALDLSLIGFYTNAAAREER
ETFLTQLTLVKNVLEEISKTDPNFKNLYDSCSRLLQIIDFYTDIVQKKYGGEEDCECTRVGGAALT
VEELGLSKAARSQVDLNQAINTFMYYYYVAQIYSNLTHNKQEFQSYEENYATILGDAIAGRLMQ
LDTEKNARINSPAVDLARGHVGPNPGGAQEVDWKATVSAIELEYDVKRRFYRALEGLDLYLKNI
TKTFVNNIDSIQTVQQMLDGVRIIGRWFTETTGDTLAQVFESFPTSTGNDSNVFTDNAPAGHYYE
KVAAEIQQGRSVGTLRPVRASQAKNIRDLIGRSLSNFQALKNIINAFARIGDMLGGEELRQMVPM
SPLQIYKTLLEYLQHSALSVGLKNLNQSEIGGQRMALAQTAEEAAQRVYLSTVRVNDALSTRWE
TEDVFFTFMLKSMAAKIFIVLGIYDMFERPEPVYKLIPTRMILGGADELEPEVIPEAAELYFRLPRL
AEFYQKLFSFRDENVQISMLPELEGIFSGLIRIIFMRPIELINIGDYSETEIRQLIKEINVIYQHFNLEY
GEQEATKKALIHFVNEINRRFGVITRTEWEKFQRIVQEARTMNDFGMMNQTNYSILPDEDGYTQ
SSQLLPSDRFISPSTQPTPKWRPALYNIDSVDVQTGMLQPNSQWDLVQKFRKQLSEMFEDPSLQQ
ELGKISYQELIRQAINELKKEHTDKIQIVSKLIQGSESLADTDVNKIFLFHETVITGLNLLSAIYVLL
NNFRNNIKGLDLDTIQKSIIEWLRETQAANVNRANLIDWLGRKHGAISEIRNPGLVIKEINMRLSM
VYPDPTTEAAAA
2. p220.2
(SEQ ID NO. 103)
GLDLDTIQKSIIEWLRETQAANVNRANLIDWLGRKHGAISEIRNPGLVIKEINMRLSMVYPDPTTE
AAAAAQDRNLTTETLFAWIVPYVGIPAGGGVRPEQELAARYLVDNQRIMQLLLTNIFEMTSSFN
KMVQVRFPETSTAQVHLDFTGLISLIDSLMADTKYFLDLLRPHIDKNIIQYYENRSNPGSFYWLEE
HLIDKLIKPPTDAGGRPLPGGELGLEGVNQIINKTYTLLTKPYNVLQLRGGAQRRDAANIQINNNP
QSSERFEQYGRVFSRLVFYDALENNSGLRVEQVALGDFRLSNLIRTNNAQEENTLSYWDNIALRT
YANVNDAANNLRRYRLYGSDYGIQNNRSMMMVFNQLIASYITRFYDAPSGKIYLNLINAFANGN
FSQAVMEMGYAHPDLARNNNVFGHRGDPTEQSVLLLSLGLILQRLIKDTNRQGLSQHLISTLTEI
PIYLKENYRANLPLFNKMFNILISQGELLKQFIQYTNVQLARPNLTALLGANNDSVIYYNNNNVP
ATGLSVGQAALRGIGGVFRPNVTLMPLGDAQNNTSDVVRKRLVAVIDGIIRGSHTLADSAMEVL
HELTDHPIYLETEEHFIQNYMSRYNKEPLMPFSLSLYYLHDLRIENNEVYDPLLYPNLESGSPEFK
LLYGTRKLLGNDPVQLSDMPGVQLIMKNYNETVVAREQITPTRFEHFYTHAIQALRFIINIRSFKT
VMMYNENTFGGVNLISENRDDKPIITAGIGMNAVYSLRKTLQDVISFVESSYQEEQINHIHKIVSP
KGQTRTLGSNRERERIFNLFDMNIIPINVNALMRSIPLANIYNYDYSFEEIACLMYGISAEKVRSLD
TTAPQPDVAEVLNIPNRPPINTREFMLKLLINPYVSVSITQYGNELLSKGNAGYMSRIFRGDNALN
MGRPKFLSDQIFNKVLFGSLYPTQFDYDEAGPSLAAGIQRGRERWGHPMSIYINQALHEIVRTIRL
AETVRGLRNVIDRNQIIGELNAFRTQLEDTRREVNNLIQTPEIQNNPTPEIIAAIQNWVQQYRGQIT
NLIDLIGNAGQANSMINLIQNITPQTAGAQLTALFNIRGLPAPPPRQALQNDIEAMQWFMTMVIN
HPPVLIAPFMLLVNNLKEFLNTLERYVYKTPRWLGPGTARIAQPPVGMAPGINMRHHTSYTENS
VLTYITEQNREEGPWSIVKQVGVGIQKPTLVQIGKDRFDTRLIRNLIFITNIQRLLRLRLNLELSQFR
NVLVSPDHIINPSITEYGFSITGPSETFSDKQYDSDIRIL
3. p72-p15-B602L
(SEQ ID NO. 104)
MASGGAFCLIANDGKADKIILAQDLLNSRISNIKNVNKSYGKPDPEPTLSQIEETHLVHFNAHFKP
YVPVGFEYNKVRPHTGTPTLGNKLTFGIPQYGDFFHDMVGHHILGACHSSWQDAPIQGTSQMGA
HGQLQTFPRNGYDWDNQTPLEGAVYTLVDPFGRPIVPGTKNAYRNLVYYCEYPGERLYENVRF
DVNGNSLDEYSSDVTTLVRKFCIPGDKMTGYKHLVGQEVSVEGTSGPLLCNIHDLHKPHQSKPIL
TDENDTQRTCSHTNPKFLSQHFPENSHNIQTAGKQDITPITDATYLDIRRNVHYSCNGPQTPKYY
QPPLALWIKLRFWFNENVNLAIPSVSIPFGERFITIKLASQKDLVNEFPGLFVRQSRFIAGRPSRRNI
RFKPWFIPGVINEISLTNNELYINNLFVTPEIHNLFVKRVRFSLIRVHKTQVTHTNNNFIHDEKLMS
ALKWPIEYMFIGLKPTWNISDQNPHQHRDWHKFGHVVNAIMQPTHHAEISFQDRDTALPDACSS
ISDISPVTYPITLPIIKNISVTAHGINLIDKFPSKFCSSYIPFHYGGNAIKTPDDPGAMMITFALKPREE
YQPSGHINVSRAREFYISWDTDYVGSITTADLVVSASAINFLLLQNGSAVLRYSTGSGATNFSLLK
QAGDVEENPGPMADFNSPIQYLKEDSRDRTSIGSLEYDENADTMIPSFAAGLEEFEPIPDYDPTTS
TSLYSQLTHNMEKIAEEEDSNFLHDTREFTSLVPDEADNKPEDDEESGAKPKKKKHLFPKLSSHK
SKGSGATNFSLLKQAGDVEENPGPMAEFNIDELLKNVLEDPSTEISEETLKQLYQRTNPYKQFKN
DSRVAFCSFTNLREQYIRRLIMTSFIGYVFKALQEWMPSYSKPTHTTKTLLSELITLVDTLKQETN
DVPSESVVNTILSIADSCKTQTQKSKEAKTTIDSFLREHFVFDPNLHAQSAYTCADTNVDTCASM
CADTNVDTCASMCADTNVDTCASTCTSTEYTDLADPERIPLHIMQKTLNVPNELQADIDAITQTP
QGYRAAAHILQNIELHQSIKHMLENPRAFKPILFNTKITRYLSQHIPPQDTFYKWNYYIEDNYEEL
RAATESIYPEKPDLEFAFIIYDVVDSSNQQKVDEFYYKYKDQIFSEVSSIQLGNWTLLGSFKANRE
RYNYFNQNNEIIKRILDRHEEDLKIGKEILRNTIYHKKAKNIQETGPDAPGLSIYNSTFHTDSGIKG
LLSFKELKNLEKASGNIKKAREYDFIDDCEEKIKQLLSKENLTPDEESELIKTKKQLDNALEMLNV
PDDTIRVDNKLEKEILYTKAEL
4. p62-p32-p54-EP153R-p10
(SEQ ID NO. 105)
MPSNMKQFCKISVWLQQHDPDLLEIINNLCMLGNLSAAKYKHGVTFIYPKQAKIRDEIKKHAYS
NDPSQAIKTLESLILPFYIPTPAEFTGEIGSYTGVKLEVEKTEANKVILKNGEAVLVPAADFKPFPD
RRLAVWIMESGSMPLEGPPYKRKKEGGGNDPPVPKHISPYTPRTRIAIEVEKAFDDCMRQNWCS
VNNPYLAKSVSLLSFLSLNHPTEFIKVLPLIDFDPLVTFYLLLEPYKTHGDDFLIPETILFGPTGWN
GTDLYQSAMLEFKKFFTQITRQTFMDIADSATKEVDVPICYSDPETVHSYANHVRTEILHHNAVN
KVTTPNLVVQAYNELEQTNTIRHYGPIFPESTINALRFWKKLWQDEQRFVIHGLHRTLMDQPTYE
TSEFAEIVRNLRFSRPGNNYINELNITSPAMYGDKHTTGDIAPNDRFAMLVAFINSTDFLYTAIPEE
KVGGNETQTSSLTDLVPTRLHSFLNHNLSKLKILNRAQQTVRNILSNDCLNQLKHYVKHTGKNEI
LKLLQEGSGATNFSLLKQAGDVEENPGPMDFILNISMKMEVIFKTDLRSSSQVVFHAGSLYNWFS
VEIINSGRIVTTAIKTLLSTVKYDIVKSARIYAGQGYTEHQAQEEWNMILHVLFEEETESSASSENI
HEKNDNETNECTSSFETLFEQEPSSEVPKDSKLYMLAQKTVQHIEQYGKAPDFNKVIRAHNFIQTI
YGTPLKEEEKEVVRLMVIKLLKKISFYLTYIGSGATNFSLLKQAGDVEENPGPMDSEFFQPVYPR
HYGECLSPVTTPSFFSTHMYTILIAIVVLVIIIIVLIYLFSSRKKKAAAIEEEDIQFINPYQDQQWVEV
TPQPGTSKPAGATTASVGKPVTGRPATNRPATNKPVTDNPVTDRLVMATGGPAAAPAAASAPA
HPAEPYTTVTTQNTASQTMSAIENLRQRNTYTHKDLENSLGSGATNFSLLKQAGDVEENPGPMF
SNKKYIGLINKKEGLKKKIDDYSILIIGILIGTNILSLIINIIGEINKPICYQNDDKIFYCPKDWVGYNN
VCYYFGNEEKNYNASNYCKQLNSTLTNNTILVNLTKTLNLTKTYNHESNYWVNYSLIKNEV
LLRDSGYYKKQKHVSLLYICSKGSGATNFSLLKQAGDVEENPGPMPTKAGTKSTANKKTTKGSS
KSGSSRGHTGKTHASSSMHSGMLYKDMVNIARSRGIPIYQNGSRLTKSELEKKIKRSK
5. K205R-A104R-CD2vΔcd-A151R-9GL-K196R-CP80R
(SEQ ID NO. 106)
MVEPREQFFQDLLSAVDQQMDTVKNDIKDIMKEKTSFMVSFENFIERYDTMEKNIQDLQNKYEE
MAANLMTVMTDTKIQLGAIIAQLEILMINGTPLPAKKTTIKEAMPLPSSNTNNEQTSPPASGKTSE
TPKKNPTNAMFFTRSEWASSNTFREKFLTPEIQAILDEQFANKTGIERLHAEGLYMWRTQFSDEQ
KKMVKEMMKKGSGATNFSLLKQAGDVEENPGPMSTKKKPTITKQELYSLVAADTQLNKALIER
IFTSQQKIIQNALKHNQEVIIPPGIKFTVVTVKAKPARQGHNPATGEPIQIKAKPEHKAVKIRALKP
VHDMLNGSGATNFSLLKQAGDVEENPGPMIILIFLIFSNIVLSIDYWVSFNKTIILDSNITNDNNDIN
GVSWNFFNNSFNTLATCGKAGNFCECSNYSTSIYNITNNCSLTIFPHNDVFDTTYQVVWNQIINY
TIKLLTPATPPNITYNCTNFLITCKKNNGTNTNIYLNINDTFVKYTNESILEYNWNNSNINNFTATC
IINNTISTSNETTLINCTYLTLSSNYFYTFFKLYYIPLSIIIGITISILLISIITFLGSGATNFSLLKQAGDV
EENPGPMNKKIIVMMALLHKEKLIECIYHELENGGTILLLTKNIVVSEISYIGNTYKYFTFNDNHD
LISKEDLKGATSKNIAKMIYNWIIKNPQNNKIWSGEPRTQIYFENDLYHTNYNHKCIKDFWNVST
SVGPHIFNDRSIWCTKCTSFYPFTNIMSPNIFQGSGATNFSLLKQAGDVEENPGPMLHWGPKYWR
SLHLYAIFFSDAPSWKEKYEAIQWILNFIESLPCTRCQHHAFSYLTKNPLTLNNSEDFQYWTFAFH
NNVNNRLNKKIISWSEYKNIYEQSILKTIEYGKTDFIGAWSSLGSGATNFSLLKQAGDVEENPGP
MNIIRKLKPGTISLVLGPMFAGKTTFLIHCIYMLERLEKKVVFIKSTKNTRDKTIKTHSGIQLRPKQ
CKIIESTQLSDVGSLTDIHAVVVDEAHFFDDLITCRTWAEEEKIIILAGLNASFEQKMFPPIVRIFPY
CSWVKYIGRTCMKCNQHNACFNVRKNADKTLILAGGSELYVTCCNNCLKNTFIKQLQPIKYGSG
ATNFSLLKQAGDVEENPGPMLIPVVCFTCGFPIGTYAAIFDKARTEYIKTKMGGTLPQNIPLDASL
QIELKDLITALGIPMRVCCRTHLITTLDYRKYY
6. B438L-R298L-NP419L-K145R
(SEQ ID NO. 107)
MYHDYASKLLADYRSDPPLWESDLPRHNRYSDNILNSRYCGNKNGAAPVYNEYTNSPEKAEKG
LQLSDLRNFSFMLNPQHKNIGYGDAQDLEPYSSIPKNKLFNHFKNHRPAFSTHTENLIRRNVVRT
EKKTFPQVASLKGTQKNCLTQPSSLPSLKNPKNSSVPSTRFSEHTKFFSYEDLPKLRTKGTIKHEQ
HLGDQMPGQHYNGYIPHKDVYNILCLAHNLPASVEKGIAGRGIPLGNPHVKPNIEQELIKSTSTY
TDVPMLGPLPPKDSQHGREYQEFSANRHMLQVSNILHSVFANHSIKPQILEDIPVLNAQLTSIKPV
SPFLNKAYQTHYMENIVTLVPRFKSIANYSSPIPNYSKRNSGQAEYFDTSKQTISRHNNYIPKYTG
GIGDSKLDSTFPKDFNASSVPLTSAEKDHSLRGDNSACCISSISPSLGSGATNFSLLKQAGDVEENP
GPMSRPEQQLKKMLKNPQAQYAFYPTAKVERISTTQHMYFIATRPMFEGGRNNVFLGHQVGQPI
IFKYVSKKEIPGNEVIVLKALQDTPGVIKLIEYTENAMYHILIIEYIPNSVDLLHYHYFKKLEETEA
KKIIFQLILIIQNIYEKGFIHGDIKDENLIIDINQKIIKVIDFGSAVRLDETRPQYNMFGTWEYVCPEF
YYYGYYYQLPLTVWTIGMVAVNLFRFRAENFYLNDILKRENYIPENISETGKQFITECLTINENKR
LSFKSLVSHPWFKGLKKEIQPISELGVDYKNVITGSGATNFSLLKQAGDVEENPGPMLNQFPGQY
SNNIFCFPPIESETKSGKKASWIICVQVVQHNTIIPITDEMFSTDVKDAVAEIFTKFFVEEGAVRISK
MTRVTEGKNLGKKNATTVVHQAFKDALSKYNRHARQKRGAHTNRGIIPPMLVKYFNIIPKTFFE
EETDPIVQRKRNGVRAVACQQGDGSILLYSRTKKEFLGLDNIKKELKQLYLFIDVRVYLDGELYL
HRKPLQWIAGQANAKTDSSELHFYVFDCFWSDQLQMPSNKRQQLLTNIFKQKEDLTFIHQVENF
SVKNVDEALRLKAQFIKEGYEGAIVRNANGPYEPGYNNYHSAHLAKLKPLLDAEFILVDYTQGK
KGKDLGAILWVCELPNKKRFVVTPKHLTYADRYALFQKLTPALFKKHLYGKELTVEYAELSPKT
GIPLQARAVGFREPINVLEIIGSGATNFSLLKQAGDVEENPGPMDHYLKKLQDIYTKLEGHPFLFS
PSKTNEKEFITLLNQALASTQLYRSIQQLFLTMYKLDPIGFINYIKTSKQEYLCLLINPKLVTKFLKI
TSFKIYINFRLKTFYISPNKYNNFYTAPSEEKTNHLLKEEKTWAKIVEEGGEES
7. B385R-F334L-CP312R-H108R-F165R
(SEQ ID NO. 108)
MDEIINKYQAVEKLFKEIQQGLAAYDQYKTLISEMMHYNNHIKQEYFNFLMIISPYLIRAHSGETL
RNKVNNEIKRLILVENINTKISKTLVSVNFLLQKKLSTDGVKTKNMWCTNNPMLQVKTAHNLFK
QLCDTQSKTQWVQTLKYKECKYCHTDMVFNTTQFGLQCPNCGCIQELMGTIFDETHFYNHDGQ
KAKSGIFNPNRHYRFWIEHILGRNPEQELGTKQDPCGTKVLQQLKKIIKRDNKCIALLTVENIRKM
LKEINRTDLNNCVSLILRKLTGVGPPQISESILLRGEYIFTEAIKIREKVCKKGRINRNYYPYYIYKIF
DAILPPNDTTNRRILQYIHLQGNDTLANNDSEWESICMELPEIKWKPTDRTHCVHFFGSGATNFSL
LKQAGDVEENPGPMLIFISNMEELLIENSQRFTIFPIQHPECWNWYKKLESLTWTAQEVDMCKDI
DDWEAMPKPQREFYKQILAFFVVADEIVIENLLTNFMREIKVKEVLYFYTMQAAQECVHSEAYSI
QVKTLIPDEKEQQRIFSGIEKHPIIKKMAQWVRQWMDPDRNTLGERLVGFAAVEGILFQNHFVAI
QFLKEQNIMPGLVSYNEFISRDEGMHCSFACFLISNYVYNIPEEKIIHKILKEAVELVDEFINYAFD
KARGRVPGFSKEMLFQYIRYFTDNLCFMMQCKSIYKVGNPFPQMTKFFLNEVEKTNFFELRPTQ
YQNCVKDDAFAFKLFLNDDDFGSGATNFSLLKQAGDVEENPGPMTTHIFHADDLLQALQQAKA
EKNFSSVFSLDWDKLRTAKRNTTVKYVTVNVIVKGKKAPLMFNFQNEKHVGTIPPSTDEEVIRM
NAENPKFLVKKRDRDPCLQFNKYKISPPLEDDGLTVKKNEQGEEIYPGDEEKSKLFQIIELLEEAF
EDAVQKGPEAMKTKHVIKLIQRKISNSAVKNADKPLPNPIARIRIKINPATSILTPILLDKNKPITLQ
NGKTSFEELKDEDGVKANPDNIHKLIESHSIHDGIINARSICISNMGISFPLCLEMGVVKVFEKNNG
IDVNSIYGSDDISTLVNQIAIAGSGATNFSLLKQAGDVEENPGPMVNLFPVFTLIVIITILITTRELST
TMLIVSLVTDYIIINTQYTEQQHENNTFFMPQKNSFNESYNKDKKSNIHIPYQWLAPELKEAESKY
WWGNYDPHSEPVLAGASGSGATNFSLLKQAGDVEENPGPMVNPNKRIMNKKSKQASISSILNFF
FFYIMEYFVAVDNETSLGVFTSIEQCEETMKQYPGLHYVVFKYMCPADAENTDVVYLIPSLTLHT
PMFVDHCPNRTKQARHVLKKINLVFEEESIENWKVSVNTVFPHVHNRLSAPKLSIDEANEAVEKF
LIQAGRLMSL
8. F778R-S273R-MGF100.1L_B66L
(SEQ ID NO. 109)
METFFIETLASDVYGKALNVDLDRLSQAQVKYTLQELISYCSALTILHYDYSTLAARLSVYQLHQ
STASSFSKAVRLQAAQSCSRLSPQFVDVVYKYKAIFDSYIDYSRDYKLSLLGIETMKNSYLLKNK
DGVIMERPQDAYMRVAIMIYGMGRVVNMKMILLTYDLLSQHVITHASPTMFNAGTKKPQLSSC
FLLNVNDNLENLYDMVKTAGIISGGGGGIGLCLSGIRAKNSFISGSGLKSNGIQNYIVLQNASQCY
ANQGGLRPGAYAVYLELWHQDIFTFLQMPRLKGQMAEQRLNAPNLKYGLWVPDLFMEILEDQI
HNRGDGKWYLFSPDQAPNLHKVFDLERSQHENAHREFKKLYYQYVAEKRYTGVTTAKEIIKEW
FKTVVQVGNPYIGFKDAINRKSNLSHVGTITNSNLCIEVTIPCWEGDKAEQGVCNLAAVNLAAFI
RENGYDYRGLIEASGNVTENLDNIIDNGYYPTEATRRSNMRHRPIGIGVFGLADVFASLKMKFGS
PEAIAMDEAIHAALYYGAMRRSIELAKEKGSHPSFPGSAASKGLLQPDLWVRCGDLVSSWEERV
AQTTQGVLTPKRWSQLRLAAMQGLRNGYVTALMPTATSSNSTGKNECFEPFTSNLYTRRTLSGE
FIVLNKYLIDDLKEINLWTEAIQQQLLNAGGSIQHILDIPAEIRDRYKTSREMNQKILTKHAAARN
PFVSQSMSLNYYFYEPELSQVLTVLVLGWKKGLTTGSYYCHFSPGAGTQKKIIRNSEKACNADC
EACLLGSGATNFSLLKQAGDVEENPGPMSILEKITSSPSECAEHLTNKDSCLSKKIQKELTSFLEK
KETLGCDSESCVITHPAVKAYAQQKGLDLSKELETRFKAPGPRNNTGLLTNFNIDETLQRWAIKY
TKFFNCPFSMMDFERVHYKFNQVDMVKVYKGEELQYVEGKVVKRPCNTFGCVLNTDFSTGTG
KHWVAIFVDMRGDCWSIEYFNSTGNSPPGPVIRWMERVKQQLLKIHHTVKTLAVTNIRHQRSQT
ECGPYSLFYIRARLDNVSYAHFISARITDEDMYKFRTHLFRIAGSGATNFSLLKQAGDVEENPGP
MGNKESKYLEMCSEEAWLNIPNIFKCIFIRKLFYNKWLKYQEKKLKKSLKLLSFYHPKKDFVGIR
DMLQMAPGGSYFITDNMTEEFLMLVVKHPEDGSAEFTKLCLKGSCIVIDGYYYDNLHIFISETPDI
YKYPLIRYDRMDIKRALILFLLFLVVLSNAFVDYIISNFNHAVTCRKPTYFGIVLQGIFLVILFSIVD
YLINENIL
9. NP868R-H339R
(SEQ ID NO. 110)
MASLDNLVARYQRCFNDQSLKNSTIELEIRFQQINFLLFKTVYEALVAQEIPSTISHSIRCIKKVHH
ENHCREKILPSENLYFKKQPLMFFKFSEPASLGCKVSLAIEQPIRKFILDSSILVRLKNRTTFRVSEL
WKIELTIVKQLMGSEVSAKLAAFKTLLFDTPEQQTTKNMMTLINPDDEYLYEIEIEYTGKPESLTA
ADVIKIKNTVLTLISPNHLMLTAYHQAIEFIASHILSSEILLARIKSGKWGLKRLLPQVKSMTKADY
MKFYPPVGYYVTDKADGIRGIAVIQDTQIYVVADQLYSLGTTGIEPLKPTILDGEFMPEKKEFYGF
DVIMYEGNLLTQQGFETRIESLSKGIKVLQAFNIKAEMKPFISLTSADPNVLLKNFESIFKKKTRPY
SIDGIILVEPGNSYLNTNTFKWKPTWDNTLDFLVRKCPESLNVPEYAPKKGFSLHLLFVGISGELF
KKLALNWCPGYTKLFPVTQRNQNYFPVQFQPSDFPLAFLYYHPDTSSFSNIDGKVLEMRCLKREI
NHVSWEIVKIREDRQQDLKTGGYFGNDFKTAELTWLNYMDPFSFEELAKGPSGMYFAGAKTGI
YRAQTALISFIKQEIIQKISHQSWVIDLGIGKGQDLGRYLDAGIRHLVGIDKDQTALAELVYRKFS
HATTRQHKHATNIYVLHQDLAEPAKEISEKVHQIYGFPKEGASSIVSNLFIHYLMKNTQQVENLA
VLCHKLLQPGGMVWFTTMLGEQVLELLHENRIELNEVWEARENEVVKFAIKRLFKEDILQETGQ
EIGVLLPFSNGDFYNEYLVNTAFLIKIFKHHGFSLVQKQSFKDWIPEFQNFSKSLYKILTEADKTW
TSLFGFICLRKNGSGATNFSLLKQAGDVEENPGPMAGRVKIKQKELIDSTVKNKNVMNLFHEIIG
SKGNINFSVVWPKFKKIKQSVYDYISTLSVLEKANVMQNFEADKKLLELFVQKLWAAYEGYFK
YPEIEKYEVEGQVNFNLVPQCVLEKFSQLYRIRINSELVTLILNSCAFMSKYNDYILKKDPYILTIT
PGLCFSPIPNFEDLNFKHLYNSDKNSQHDKEFIMFILYKLYTAALGVYNAISIPDIDVEDLENIILSS
VSQIKKQIPRCKDAFNKIESSVHLLRKNFNTYYSDYVGSGYNPTIIMEQYIKDISQDSKNISPRISY
QFRTIIKYYRDMIATRHQTMDPQVLNLVKHVEKKLDMLDREKN
10. I329L-A224L-MGF505.6R-B175L
(SEQ ID NO. 111)
MLRVFIFFVFLGSGLTGRIKPQVTCKYFISENNTWYKYNVTILNSSIVLPAYNTIPSNAAGISCTCH
DIDYLQKNNISIHYNTSILKTFQDIRIIRCGMKNISEIAGGFGKELKFLDLRYNDLQVIDYNILRKLI
RSNTPTYLYYNNLMCGKRNCPLYYFLLKQEQTYLKRLPQFFLRRINFSNNNTFLYHFLSCGNKPG
HEFLEYQTKYCRTKFPEINITVNQLIAKKNTERYKSCYPLVFISILCSCISFLFLFICLLRSICKKYSC
TKQDKSSHNYIPLIPSYTFSLKKHRHPETAVVEDHTTSANSPIVYIPTTEEKKVSCSRRKGSGATNF
SLLKQAGDVEENPGPMFPKINTIDPYISLRLFEVKPKYVGYSSIDARNQSFAIHDIKNYEKFSNAGL
FYTSPTEITCYCCGMKFCNWLYEKHPLQVHAFWSRNCGFMRATLGIIGLKKMIDSYNDYYNNEV
FVKHQNRVYTHKRLEDMGFSKPFMRFILANAFIPPYRKYIHKIILNERYFTFKFAAHLLSFHKVNL
DNQTTYCMTCGIEPIKKDENFCNACKTLNYKHYKTLNFSVKLGSGATNFSLLKQAGDVEENPGP
MFSLQDLCRKNIFFLPNDFSKHTLQWLGLYWKEHGSVHRAEKDSIMIQNELVLSINDALQLAGE
EGDTDVVQLLLLWEGNLHYAIIGALKTEKYNLICEYHSQIQDWHILLPMIQDPETFEKCHDLSLG
CDFICLLQHAVKYNMLSILVKYKEDLLNARIRHRIQSLFVLACENRRIEIIDWIGQNLPIPEPDAIFS
IAVATRDLELFSLGYKIIFDYMQRQGIIQLTNGVRMVVLNRHISMAIDNGLLPFVLETLKHGGNIH
RALSYAVTHNRRKILDYLIRQKNIAPNTIERLLYLAVKNQSSRKTLNLLLSYINYKVKNVKKLVE
HVVNEKSTLVLKILLEKKENLVDAVLTRLVKHSTYFQVREFIQEFSISPEKFIKIAVREKKNVLIEA
ISEDIWENPTERITYLKQIVHTIKYESGRRFLVDIIHSIYQSYSLKHEDILKLATFYVKHNAITHFKD
LCKYLWLNRGTESKKLFLECLEIADEKEFPDIKSIVSEYINYLFTAGAITKEEIMQAYDALEGSGA
TNFSLLKQAGDVEENPGPMETNCPNILYLSGITIEECLQSKKTATDTLNTNDDEAEVEKKLPSVFT
TVSKWVTHSSFKCWTCHLYFKTVPKFVPTYMRENERGEIEMGVLGNFCSFSCAASYVDVHYTEP
KRWEARELLNMLYRFFTSQWISYIKPAPSYTMRKEYGGKLSEEAFISELHTLEESISSKHIFI
11. C475L-B354L-D345L-H124R
(SEQ ID NO. 112)
MTSLLKTDFNVSKYRLIAQKREANAVEIEAALEVVREFIIKKKLILYGGIAIDYALHLKGSSIYPEG
ERPDFDMFSPNHVEDAYELADILYEKGFKQVGTVRAIHVQTMRVRTDFVWVADLSYMPPNIFNT
IPTLTYKNLKIIHPDYQRAGLHLAFCFPFDNPPREDVFSRFKKDLQRYNLIEKYYPIPVVPVKSTYE
SKTFSIPFKQVAIHGFAAYALLYQTLNELRITCKVPEWKTEFPQPSYSYHKNDKNITLTVDMPKA
YPALVLATYNPEEVIKEMGLHLTEICEPYMDYSPPIFKTNDIHFFSTMFKELAISIIQDNLIVVSPQY
LLLYFLYGAFATPADKSLFLFYYNATLWILEKADSLLNIIQKQTSPEEFTRFANTSPFVLTTRVLSC
SQERCTFSPAYRISLANDVQQSQLPLPKTHFLSNSLPDVSTLPYNYYPGKGKDRPTNFSYEKNLLF
NIGGKCTPSAMGSGATNFSLLKQAGDVEENPGPMALTTHSGKLIPELQFKAHHFIDKTTVLYGPS
KTGKTVYVKHIMKILQPHIEQILVVAPSEPSNRSYEGFVHPTLIHYRLWLADKQKKNDNKGAERF
LEAIWQRQTMMSSIYSRVNNIDMLKTLYHKLPIDIQQKENKNIAKVECLKAEQTDQKKEEKITSL
YQQLLKKIIIQNIHMYKNLCLTEDEKFTLNYINLNPRLLLILDDCAAELHPLFTKEIFKKFFYQNRH
CFISMIICCQDDTDLPANLRKNAFVSIFTNASICMSNFSRQSNRYSKQDKEYVEEISHIVFKGYRKL
VYIREDEHRQHFYHSTVPLPTAFSFGSKALLKLCKAVYSKEVVIDKSNPYWSKFRLNFGSGATNF
SLLKQAGDVEENPGPMETFVRLFKDSPQQRSDAWHAIRRTQVGGSDLASVLGLNPYKSYYITLA
EKANLFKKNLNRAACSWGTLFERVSKDLLELFCQTTVIGDNIHIDGTYLGYPGHSNSPDGFCHLT
LGYTQQSWEIKTIFNNVRYEATKRIPVLVEIKSPFNRKIKNSVPSYYMPQIQSGLALSPPISMGIYV
EAMFRVCGIHQLGSNNETNTDIHPPESMLPLAWGIITICSTQEHTEAPQDFGTLDAETFRQLLETL
YQKDQYTIHYSMPYETACPEMPNVVGYFGWKVFIFQIIPVMKHPQFLKDKYPIIQQFLRDLHTIK
ASPSPMEMYEKICCSEESALSTEDIDNFTDMLTGSGATNFSLLKQAGDVEENPGPMNLEYVQVV
QKFNQVLLELTKKVCTVVGGSKPTYWYHHIRRVCSECPSMPMSMIGPYLNVYKAQILTRDKNFF
MNFDPAHNEYTFIIQKLKEAARNMPEDELEQYWVKLLFLLKSYIKCKPFIN
12. C315R-C147L-MGF505.7R-MGF300.IL
(SEQ ID NO. 113)
MDALLKEIEKLSQPSLQKENNDVCDLCFMQMKKISNYQLLCEECGQLKDWFEPEYNEKFTVYSR
LKIVGANSSYHQRDLDKANSSDYSSLQFHHILEELKSLNVKYMDAGQKPFPIQVLKETAHSYNQ
VQQHRVIRSITKLQILASILRSICLKLNIACTVADAARFTQLNTKGISRGMDLLRSLFVDNKITLNV
DLNPIDSFINSTYSALQIKQIHQELQEENVYNLKEIVKSFILYADEKNIGVDLNRRTVVIATMYNVL
RRAYYPIEIDTVVYQCKIRKNTITRALKMYEDYYSHFKSLYEQYHLNAAKKLIGSGATNFSLLKQ
AGDVEENPGPMADNDNEDLIMDDLVEEYVETEEENLVDSEEESEDKDEIVESPSICEGFVQASSQ
TLVIIPDNERITSNVLTTFEATRLVAVRAQQLAINGSTMLKKKYSSPIDIAKQELFNRKIPLLVMRC
IKVTPEGQKIVEIWNPREMGIPLLDGSGATNFSLLKQAGDVEENPGPMFSLQDLCRKNTFFLPSDF
SKHTLHLLGLYWKGHGSIQRIKNDGVLIEHDLTLSINEALILAGEEGNNEVVKLLLLWEGNLHYA
IIGALRTENYNLVCEYHSQIQDWHVLLPLIQDPETFEKCHDLSLECDLSCLLQHAVKYNMLSILV
KYKEDLLNVLFRQQIQGLFILACENRKLEILTWMGQNLPIPDPEPIFSIAVVTKDLEMFSLGYKIVF
EYMENQGLHLTQVVRMVMLNHHFGMVINKGLLPFVLEILNYGGNVNRALSYAVTQNKRKILD
HVVRQKNIPHKTIERMLHLAVKKHAPRKTLNLLLSYINYKVKNVKKLLEHVVKYNSTLVIRLLL
EKKKNLLDATLTRYVKDSTYFQVKEFMQDFSISPEKFIKIAVREKRNVLIKGISEDIWENPAERIR
NLKQIVCTIKYESGRQFLINIIHTIYQSYSLKPEEILKLATFYVKHNATTHFKDLCKYLWLNRRTE
SKKLFLECLEIADKKEFPDIKSIVSEYINYLFTAGAITKEEIMQAYALEYAMYGSGATNFSLLKQA
GDVEENPGPMVSLTTCCLKNIVNQHAYVENTVLLYHLGLRWNCKTLYQCTQCNGVNYTNSHS
DQCKNKDLFLIKVIVKKNLAVARTLLSWGASPEYARLFCRNTEEEQALNVQHVADVPSSKILER
LTMSYKGNDEQLLITFYLLNLSTNFSTNLREQVRFKIVSYIICDLAIHQTFKIFYAKNYSLSTLYCIF
LAIYYKLYTALRKMVKIYPGLKSFAYLTGFMFDDETVMETYNSTDDEISECKNRIITIKGYYGNIH
CRSDIDHMYAFSQNNFW
13. MGF360.6L-MGF360.12L-MGF300.4L-D205R
(SEQ ID NO. 114)
MNSLQVLTKKVLIENKAFSNYHEDDIFILQQLGLWWENGPIGFCKQCKMVTSGSMSCSDVDSYE
LDRALVRAVKKNQTDLIKLFVLWGANINYGIICAKTERTKVLCIQLGADPKFLDVGLYNMFVDLI
KQQKVLLAIDIYYDNISILDSFDSHDFYVLIDFIYNRFILNLDEKEKMIKNTYVLKFWFKIAIEFNLI
KPIRFLSKKFPHLDYWRLKTAVYLGNVDEIHHAYFQENIRLDPNDMMSLACMYPQNKLGIYYCF
ALGANINTALETLIGFINHEVNREITFFSNYGIWSNVHFCISLGANPYTKKIQETLLRQEKNVMMK
LLFKKGLLSPHSILHKKILEPSEVRKIISTYEYTETFHSFSSLRDNLRGSGATNFSLLKQAGDVEENP
GPMLPSLQSLTKKVLAGQCVPTNQHYLLKCYDLWWHDAPITFDHNLRLIKSAGIKEGLNLNTAL
VKAVRENNYNLIKLFAEWGADINYGLVSVNTEHTWDLCRELGAKETLNEEEILQIFIDLKFHKTS
SNIILCHEVFSNNPILQKVNNIKMRIEIFWELRELIVKTDLLNNEFSLSTLLLKYWYAIAIRYNLKE
AIQYFYQKYTHLNTWRLTCALCFNNVFDLHEAYEKDKIHMDIEEMMRIACIKDHNLSTMYYCY
VLGANINQAMLSSIQYYNIENMFFCIDLGADVFEEGTTALGEGYELIKNILSLKIYSPATTPLPKST
DPEIIDHALKNYVSKNMMIFLTYDLRGSGATNFSLLKQAGDVEENPGPMQSLFNIALKALTLKNH
VEFLKRDKEVLTRLGLCCKNYDLIHKCSECGNICPNGQQHGTCININYLLIYAVKRDNYMLAYR
LLCWGANEKFAHYFRRPLPNLKPLLPKKELTPKDIKQLAYEHFHSDSELITVFEVFRKSRNINDCL
EFFYKKNIEFEIYFARLYVYSKTFYRKSWYWFCIFMAVKHSMEHALKKITKTYIPTFYNKTTLPL
VLFLSACFYENVEWMKNFFYKANKKIQQKMLSYGMEWAATHGKVRTFVCCYTLGGTASLKM
YQKAYQNERYMIMALCSYLGNIQINNPWDSLNPYMMVQNKEKFLPLKFSEETQYFYIGSGATNF
SLLKQAGDVEENPGPMDTAMQLKTSIGLITCRMNTQNNQIETILVQKRYSLAFSEFIHCHYSINAN
QGHLIKMFNNMTINERLLVKTLDFDRMWYHIWIETPVYELYHKKYQKFRKNWLLPDNGKKLIS
LINQAKGSGTLLWEIPKGKPKEDESDLTCAIREFEEETGITREYYQILPEFKKSMSYFDGKTEYKHI
YFLAMLCKSLEEPNMNLSLQYENRIAEISKISWQNMEAVRFISKRQSLNLEPIIGPAFNFIKNYLRY
KH
14. MGF360.4L-MGF360.15R-A238L-H240R-B125R
(SEQ ID NO. 115)
MNSLQVLTKKVLIETKAFSNYHEDDIFILQQLGLWWENGPIGFCKQCKMVTGGSMSCSDVDSYE
LDRALVKAVKENQTDLIKLFVLWGADINFGIMCAKTKQTKDLCIQLGANPEFLDVGLYNMFVY
LVKRKKVLLAIEIYYDNILILDSFNSNDFHLLIDFVYNRFILYLDEKEEEMTRNTLVLKFWYKFAI
DFNLTKPIHYLSKKFPHLDLWRLQTAIYLGNIDEVHHAYFQENIRLGLNVMMFLACARPGNKLGI
YYCFALGADLDRALERLISFNSINREINRKIRGEKRLCIEGSYLSNVYFCIGLGANPYTKKIQEIIKQ
KHSNIMILLFSKKKILSPHSVLQNKILDPSDVHKMISTYKNTESFYPFSSLAVKLIQQANIGSGATN
FSLLKQAGDVEENPGPMNKIFLNITETINMVLIEFLTGFFYLYGKRLFSISKVMDMICLDYYTIIPA
PLAMMLAARLKNYDLMKRLHEWEISIDYALLVVDDVPSIDYCLSLGARSPTRAQKRELLRDNTF
NPVYKYLMNCSGFPTKREKNIPCDVQCERLQKNIIKELVFNCSVLLEMVLHTEREYAYALHCAA
KHNQLPILMYCWQQSTDAESILLKTCCSDKNINCFNYCILYGGAQNLDAAMVEAAKHDARMLI
NYCVMLGGRSLNEAKETAAMFGHIECAQHCFKLQSYVVDTSNTDDTDGSGATNFSLLKQAGDV
EENPGPMFPEREIENLFVKWIKKHIRNGNLTLFEEFFKTDPWIVNRCDKNGSSVFMWICIYGRIDF
LKFLFEQESYPGEIINPHRRDKDGNSALHYLAEKKNHLILEGVLGYFGKNGTRICLPNFNGMTPV
MKAAIRGRSLNMLSLIKFGADPTQKDYHRGFTAWDWAVFTGNMELVKSLNHDYQNLSTCISLF
TSWMFSTGGLRKSPKLLLLAVNIMSMKNFLNRIQSSCVGSGATNFSLLKQAGDVEENPGPMAAN
IIATRAVPKMASKKEHQYCLLDSQEKRHGHYPFSFELKPYGQTGANIIGVQGSLTHVIKMTVFPF
MIPFPLQKTHIDDFIGGRIYLFFKELDMQAVSDVNGMQYHFEFKVVPVSPNQVELLPVNNKYKFT
YAIPVVQYLTPIFYDLSGPLDFPLDTLSVHVDILSNHIQLPIQNHNLTTGDRVFISGYKHLQTIELCK
NNKIFIKNIPPLSSEKIKLYILKNRIRIPLYFKSLKTSKGSGATNFSLLKQAGDVEENPGPMAVYAK
DLDNNKELNQKLINDQLKIIDTLLLAEKKNFLVYELPAPFDFSSGDPLASQRDIYYAIIKSLEERGF
TVKICMKGDRALLFITWKKIQSI
15. NP1450L
(SEQ ID NO. 116)
MKFYYVFSKNMEAGYAEIAAVQFNIAGDNDHKRQGVMEVTISNLFEGTLPAEGGIYDARMGTTDH
HYKCITCSHQRKQCMGHPGILQMHAPVLQPLFIAEIRRWLRVICLNCGAPIVDLKRYEHLIRPKRLIE
AASSQTEGKQCYVCKAVHPKIVKDSEDYFTFWADQQGKIDKLYPQIIREIFSRVTYDTVVKLGRSKN
SHPEKLVLKAIQIPPISIRPGIRLGIGSGPQSFFIDINNVIQYLVRKNLLIPKDLQIVRGQKIPLNIDRNLQT
IQQLYYNFLLDSVSTTATQGGTGKRGIVMGARPAPSIMRRLPRKEGRIRKSLLGSQVWSISRSTICGN
SDLHLDEVGYPISFARTLQVAETVQHYNINRLMPYFLNGKRQYPGCSRVYKQITQSVHDIEGLKQDF
RLEVGDILYRDVVTGDVAFFNRQPSLERSSIGVHRIVVLENPKISTFQMNVSACAWYNADFDGDQM
NLWVPWSVMSRVEAELLCSVRNWFISTKSSGPVNGQVQDSTVGSFLLTRTNTPMGKNVMNKLHA
MGLFQTTQTDPPCFANYSPTDLLDGKSVVSMLLRQTPINYQRAPTWYSEVYAPYMHYNKQDISTQI
RNGELIEGVLDKKAVGAGSSGGIYHLISRRYGPQQALKMIFATQQLALNYVRNAGFTVSTADMLLT
PEAHQEVQEIINELLLESEEINNRLLHGDIMPPIGLTTHDFYEKLQLNALKFPDRILKPIMNSINPETNG
LFQMVATGAKGSNPNMIHIMAGIGQIEINTQRIQPQFSFGRTLVYYPRFALEAQAYGFICNSYIAGLT
SPEFIFGEMNGRFDLINKALSTSSTGYANRKAIFGLQSCIVDYYRRVSIDTRLVQQLYGEDGLDARQL
ETVRFETTMLSDQELEDKFKYTGIQSPLFEEEFSRLKKDRDKYRQIFLNVENFNFSQLLTDVRQVPVN
VASIVKNILLSSTSGVLPFDEKSILQKYAMVKTFCKNLPYVFINNIQERLQTPIPVYLKRAASLMRMLI
RIELATVKTLNITCEQMSAILDLIRLQYTQSLINYGEAVGILAAQSVSEPLTQYMLDSIIHRSVAGGTN
KSGIVRPQEIFSAKPVEAEQSSEMLLRLKNPEVETNKTYAQEIANSIELITFERLILQWHLLYETYSST
KKNVMYPDFASDVEWMTDFLENHPLLQPPEDIANWCIRLELNKTIMILKSISLESIINSLRAKHPNTY
IMHSVENTASGIPIIIRIYLRESAFRRSTNTRMATDEKIAVNVVDKLLNSTIRGIPGIKNANVVKLMRH
RVDAQGKLVRLDNIYAIKTNGTNIFGAMLDDNIDPYTIVSSSIGDTMELYGIEAARQKIISEIRTVMG
DKGPNHRHLLMYADLMTRTGQVTSLEKAGLNAREPSNVLLRMALSSPVQVLTDAAVDSAVNPIYG
IAAPTLMGSVPRIGTMYSDIIMDEKYITENYKSVDSLIDML
16. G1340L
(SEQ ID NO. 117)
MDFQNDFLTNPLRVTLYNPVENEYTKTFIFLGSVPANVLQACRKDLQRTPKDKEILQNFYGEDWEK
KLSQYVVGGDSDDLDEFEKLFVEDRGEETNVMMPEIETMYSEYSIFPEDTFKDIREKIYVATGIPPYR
QHIFFFQNNALQVTYRLLLSGSGVALDIRDYKKEFQQVGGVNIDASMESQKDELYVEALDSFQLIK
NIHHIFVADLNTLVAPMRRQISIAIEDNYQFDLLYYGLIMKYWPLLSPDAFKLLVQSPLQMEKQYPA
LSPSLTSLKKRLLLEQKLINFTYARAQQVIAKYEGNRLTRGTLAVTSAMIKISPLVNIQINVRNVFDLF
PATPDIPQLVVFFYSKTGPTVVSKHHITSTEPEKFSNKTFRVPTIILIRFINKKAFILTIQNNGHYFIESN
WSENERHDFNSVVSTLNNFINPIIHTINDMGPAAFPRGGSLPLPSNEDIQISISSMSVSTFWPYTLSSKG
FTELKSRWREYEQAGIISVRGLQQTGVYNFLFKKGIYSYDPHEIERMIIISSGPGRKMDINVALLQNT
YAYLFDTNVAARWETIYGGRNIRIYHRVTDIKIEMFNITQEEFNYLWVYLFVFLDNLITGPDKILVNK
LSQLHDKQQGKGASQLRALQEQDPDLYDLRKYDTQATVYSVLCQHPRPPVIYSEAEVKSMPPAKR
KELVKYWNFTEGVPAYYSCPHPDYPHLSLLEGRHPLNYCLPCCQKTKALLGTKRFYINNTCLTKHT
FVEQDLEDLNTQTSRHTLSYGKKIPVNRIAFLPHQIADELFLNTIKEPDIFCIVGVEQTMLGISNAGLF
YSLARILDLAPKALAIEIAKAANTPQYYILGNGAGNMFSSGAELANLILQTFVEQKNQLLQWDTTW
QDIFLDLVAICYDLHCVFFKDKQGDIGFEVSPSTIQKILSPSKKIAIIFDTDEGIYPMAITQQKRFLKNSE
AQYIFTEDDPVMEVIQSMSEFMCKDNWWDIHDVKNIPGYTVGKKLINRHNFCYALLIDSDTDRPIYF
PIRLSSYIHDDIPIDFDLRPTQIASFEETWKFITLFNKQYKQYEIVPSAVLQNIKKEFVGFLSEGKTGLY
FYYAPTQTLPATLEKLPIATLTIDPRDIDQAILYPLEEPYPQQNKANKAFYINHLYKFLLIEFFDVLYG
LQSNSTRKHIENLFQKTDFQKITSVTEFYTKLSDFVDLNDIHTIKHILETTDAEHALKVLQKNIFNFDY
TLLSPLQSYTYDELCQHLKKLLTPRIEFYEDIETIDRGLINIYTSCQYSTLNQPQCKKKRLRIPVNHFE
NYIHILAADILNPLKHSTLLLTGLGVIDDLQFILRPQEIISVKNKF
17. M1249L-A118R-I73R
(SEQ ID NO. 118)
MEEVITIAQIVHRGTDILSLNNEEIEALVDEIYSTLKGSNDIKNIRLIDFLFTLKDFVNHVRAEQSKLPD
LSMPIEAYIRQLLVDPDVVPIVSEKKKELRVRPSTRKEIFLINGTHLAVPAEAPIEIYGLKLRLKTFSPQ
CFMRMAEIGSFSPETLGYVASGANLTNFIRVFMKCVDQETWKKNGEGVVVTTKENIIQFTHQYIEL
YKFLRSGGHSWLINRLAEEMVHRKLDREDQGSHISNIVETEEIEPEENIKRVIFFLKELSTMYSVSPVF
TSGYMPLLYDLYRAGYLEVLWNPVEQKFLQHAEQREKEQMILQQVDMKLTEVITQARQYFKIMEE
KIGRVQSDAIREILTMEGKVDDPNSILQEVIKACGKQEAELITTEYLNIKKQWELQEKNACAHLKLV
KQLRSGLQYAELLKVLESIRVLYKEKNNTTNWNLCKACGFKLLCPHVDMLIQLQAAEASYDTMRT
KLMKFSGINKEKENNQGLIYSYFCKICGEELAHFIQEDRTADVGIIGDLNSKLRVFIWQETMKACTFI
HFGKLVDVKQFANIAVNVCLPLVYSIENIKKEEDYDPLTQLYAVIYIYAYILNLIYSSQKNKEFLTITI
HGMKADSSLNAYVTFLLEKMMQQYSGIINQLSEITDQW1ANNFREAFKKIIHQNGLQGLSVQDDTK
VLLTEILLDPMYDYAATVARIDGSIPMHKPRTPKEAEYEFKTVIGRTPAELLSQKEFYDKIYTSKYRP
DFTQLTRLNDIYFQEESLRVWWGGRDEEKTSTLIYLRAYELFLKYLQNAPNFNSELAEFKTYENAY
GEQKALLAQQGFYNIFDPNTGRADQRTRLFEYKRLPISTLYDERGLPHKWTIYVYKAVDSSQKPAEI
EVTRKDVIKKIDNHYALADLRCSVCHVLQHEVGQLNIKKVQTALKASLEFNTFYAFYESRCPKGGL
HDFQDKKCVKCGLFTYIIYDHLSQPELVHDYYNNYKDQYDKEKMSIRSIQIKKDMTTPSTETQPKPP
QEPWTFDYGKIIKTAKILDISPAVIEAIGAMEGRSYADIREGQGAPPPPTSMDDPRLMAVDSAVRIFL
YNYNCLRHVSTFNKPPIHVERLVKHLSYEEKEDLEKVLPNVVNEYHTTFKHLRVTDPASALLYSIEF
LCISFLTLYEIKEPSWVVNIVREFALTELNTIIQSEKLLSKPGAFNFMIFGEDFVCSGEDSSMDDISAYS
SPGLFGEDIIDRLDDPFSIEDVDISLDVLDNLAPQMHSNVSFNFIACVLFPTPLIPSMAMSIPRMINKRK
KRIQFLTFLTNLFLYNIVQHCISGIMETQKLISMVKEALEKYQYPLTAKNIKVVIQKEHNVVLPTGSIN
SILYSNSELFEKIDKTNTIYPPLWIRKN
18. EP1242L-I9R-C62L
(SEQ ID NO. 119)
MEPLRPQITYGPIETVDNEELTEADMLSFISAAVNSTGLIGYNIKSFDDLMDNGIPQIVKQMFNVDIT
YKDQRDHTEIDKLRESVQIQFNFTDVNIERPQHRNYSQGNKINLLPNKARLCGLSYSGPVNLAAEVI
LTAHYSNGRQEVKRASIPPFQVSTFPIMRGSNRCHTHHLSKTAKKEIGEDPNEPGGYFIARGGEWVV
DLLENIRFNTLHIHYHTMQQGNNEIIRGEFISQPGGAFENSSQIIIRYMTTGAMEINSTKFSKLRIPWY
LIFRMFGMTGDDSIIEQVVFDLESNSLVNTFMIEILEKSIHVLDPIFQPVQHELNREKIIQFLSEKVSKF
VSNPSAYKSDENAVQYLNERQLTILDKILLPHMGQTADTRVRKLRFLGLLIHKILLVIMNVFPPTDR
DSYRTKRVHGSGVSLAKAFKAIFNTSVIAPIINGFKELLKQTAFEELTQRNIIEAFSAALSKNTASDLN
RSMEQSIISGNKTIMVRQRPIVNRVSTQSLERKNLLNTISALRTVNTHNTTNASKQTERADMMRRVH
ASYPGYICVAQSADTGEKVGMSKQLAITANVCTAGEVLSLKQRLLSDPAIQQLADVSNKDIVRKGL
ARVFINGEWIGCCTNAFELAQRYRMLRREGKVVHPHTTIYWDSMVDEVEFWLDVGRLTRPLLIVD
NNIEKYNQACYKAAEARKKGDKDWEKHKIPFIQNTRFTPQMAKDILAGTLTLEDLVAQGICEFITPE
EAENCLVAFSIIELRKHKFIDVTRRFTHVDVPQAILGLAALVSPYANCTQPARVTYETNQGRQTGGW
YCFSWPYRVDMNRFFQFYNEMPLVKTIAHNYVIPNGLNTIVAYMIYGGYNQEDSVIVSQSFIDRGGF
AGTFYREEKVELESDIESFGKPDPLITKNLKPGANYEKLVDGFVPVGTVVKKGDIIIGKVAKIRGEKD
ELNKYIDRSVMYGFDEPAVVDAVMRPHGPNDEIFGLMRLRYERNLNIGDKMSSRSGNKGIAALALP
TSDMPFTEDGLQPDLIVNPHSFIPSRMTNGQMIETTVGLANALQGVVTDGTAFLPINVQLLSERLAQE
GLRFNGCQKMFNGQTGEYFDAAIFIGPTYHQRLQKFYLDDRYAVASYGPTDALTGQPLDGKRSHG
GLRLGEMEHWVLTAQGAMQTIIEKSFIDDSDGCISYICRNCGEPAIYNASHPIYKCMNCDVQADIGM
VDSRRSSIVFQHEMRAANVNITSVLSPRVFQPAMGTFSVTASAKNDNAVCKYLKEPMVENKNYKNI
LRNEFIDKKNLNDALRQHITVHNPVVDWCNNYSTFSSQDFEEYKIYIHSDLMDGRPSPKKTWCVILM
NWGSISSGTPGLFVESIRNTPSVVKINVIFLKVISNTAVSVFWRDRRIRFESDWLNSYFQK
19. G1211R-I7L-L83L
(SEQ ID NO. 120)
MISIMDRSEIVARENPVITQRVTNLLQTNAPLLFMPIDIHEVRYGAYTLFMYGSLENGYKAEVRIENI
PVFFDVQIEFNDTNQLFLKSLLTAENIVYERLETLTQRPVMGYREKEKEFAPYIRIFFKSLYERRKAIT
YLNNMGYNTAADDTTCYYRMVSRELKLPLTSWIQLQHYSYEPRGLVHRFSVTPEDLVSYQNDGPT
DHSIVMAYDIETYSPVKGTVPDPNQANDVVFMICMRIFWIHSTEPLASTCITMAPCKKSSEWTTILCS
SEKNLLLSFAEQFSRWAPDICTGFNDSRYDWPFIVEKSMQHGILEEIFNKMSLFWHQKLDTILKCYY
VKEKRVKISAEKSIISSFLHTPGCLPIDVRNMCMQLYPKAEKTSLKAFLENCGLDSKVDLPYHLMWK
YYETRDSEKIADVAYYCIIDAQRCQDLLVRHNVIPDRREVGILSYTSLYDCIYYAGGHKVCNMLIAY
AIHDEYGRIACSTIARGKREHGKYPGAFVIDPVKGLEQDKPTTGLDFASLYPSLIMAYNFSPEKFVAS
RDEANSLMAKGESLHYVSFHFNNRLVEGWFVRHNNVPDKMGLYPKVLIDLLNKRTALKQELKKL
GEKKECIHESFIPGFKELQFRHAMVDAKQKALKIFMNTFYGEAGNNLSPFFLLPLAGGVTSSGQYNL
KLVYNFVINKGYGIKYGDTDSLYITCPDSLYTEVTDAYLNSQKTIKHYEQLCHEKVLLSMKAMSTL
CAEVNEYLRQDNGTSYLRMAYEEVLFPVCFTGKKKYYGIAHVNTPNFNTKELFIRGIDIIKQGQTKL
TKTIGTRIMEESMKLRRPEDHRPPLIEIVKTVLKDAVVNMKQWNFEDFIQTDAWRPDKDNKAVQIF
MSRMHARREQLKKHGAAASQFAEPEPGERFSYVIVEKQVQFDIQGHRTDSSRKGDKMEYVSEAKA
KNLPIDILFYINNYVLGLCARFINENEEFQPPDNVSNKDEYAQRRAKSYLQKFVQSIHPKDKSVIKQG
NVHRQCYKYIHQEIKKKIGIFADLYKEFFNNTTNPIESFIQSTQFMIQYFDGEQKVNHSMKKMVEQH
ATASNRAGKPAGNPAGNALMRAIFTQLITEEKKIVQALYNKGDAIFIDLLTYIINNINYKIATFQTKQ
MLTFEFSSTHVELLLKLNKTWLILAGIHVAKKHLQAFLDSYNNESPSRTFIQQAIEEECGSIKPSCYDF
ISMGNHPIKQDMKTCDYYHGEKKLKYMRRMLYNEPLGTYAVSSLFGCDMIVLTWNCTISGRTLHR
RIHTRFGQYYHNNCYYTKIDDIIGDYPDTFYRPLYRYKPMDTSLKNNDGALEADNKNYQDYRAEP
DKTNDVLDVTKYNSMVDCCHKNYSTFTSEWYINERKYNDVPEGPKKAVVHRCTIL
20. P1192R-EP152R-E66L
(SEQ ID NO. 121)
MEAFEISDFKEHAKKKSMWAGALNKVTISGLMGVFTEDEDLMALPIHRDHCPALLKIFDEIIVNATD
HERACHNKTKKVTYIKISFDKGVFSCENDGPGIPIAKHEQASLIAKRDVYVPEVASCHFLAGTNINK
AKDCIKGGTNGVGLKLAMVHSQWAILTTADGAQKYVQHINQRLDIIEPPTITPSREMFTRIELMPVY
QELGYAEPLSETEQADLSAWIYLRACQCAAYVGKGTTIYYNDKPCRTGSVMALAKMYTLLSAPNS
TIHTATIKADAKPYSLHPLQVAAVVSPKFKKFEHVSVINGVNCVKGEHVTFLKKTINEMVVKKFQQ
TIKDKNRKTTLRDSCSNIFIVIVGSIPGIEWTGQRKDELSIAENVFKTHYSIPSSFLTSMTKSIVDILLQSI
SKKDNHKQVDVDKYIRARNAGGKRAQDCMLLAAEGDSALSLLRTGLTLGKSNPSGPSFDFCGMIS
LGGVIMNACKKVTNITTDSGETIMVRNEQLTNNKVLQGIVQVLGLDFNCHYKTQEERAKLRYGCIV
ACVDQDLDGCGKILGLLLAYFHLFWPQLIIHGFVKRLLTPLIRVYEKGKTMPVEFYYEQEFDAWAK
KQTSLANHTVKYYKGLAAHDTHEVKSMFKHFDNMVYTFTLDDSAKELFHIYFGGESELRKRELCT
GVVPLTETQTQSIHSVRRIPCSLHLQVDTKAYKLDAIERQIPNFLDGMIRARRKILAGGVKCFASNN
RERKVFQFGGYVADHMFYHHGDMSLNTSIIKAAQYYPGSSHLYPVFIGIGSFGSRHLGGKDAGSPR
YISVQLASEFIKTMFPAEDSWLLPYVFEDGQRAEPEYYVPVLPLAIMEYGANPSEGWKYTTWARQL
EDILALVRAYVDKDNPKHELLHYAIKHKITILPLRPSNYNFKGHLKRFGQYYYSYGTYVISEQRNIITI
TELPLRVPTVAYIESIKKSSNRMTFIEEIIDYSSSETIEILVKLKPNSLNRIVEEFKETEEQDSIENFLRLR
NCLHSHLNFVKPKGGIIEFNTYYEILYAWLPYRRELYQKRLMREHAVLKLRIIMETAIVRYINESAEL
NLSHYEDEKEASRILSEHGFPPLNHTLIISPEFASIEELNQKALQGCYTYILSLQARELLIAAKTRRVEK
IKKMQARLDKVEQLLQESPFPGASVWLEEIDAVEKAIIKGRNTQWKFHMYSILIACLVLLLCLVIYV
GHRADHARKYLEGMWHGDPVFLKQSGLQSFYLYIQPDHTCFFSIVNKNGEKLMETKIPCTITNKIY
MFFKPIFEFHVVMEDIHSYFPKQFNFLLDSTEGKLILENNHVIYAVLYKDNFATALGKTVEKYITQN
MHISIITRYTLKYIYMHLTHNHILFTYIMRIYLIKHNHMLFTTHVYTYIM
21. D1133L-E165R-C122R
(SEQ ID NO. 122)
MAYPELDAADFLQQLARRKEFKSLISPPVDQKELIRDLRAHFVQIGGPGCEKGGRAFFPCDPYASPF
PSIKGLQLHNAQLFVQNFQNPNTPYSRLLLNWQTGTGKSIAAIAIARQFMNHYMNFIENAPWIFVVG
FIRAIIQTEMLRRPELGFVSYKEVAELHRLLHIAKQSGSTTSVESRHLNGFVSTLKRRLTDRNRGGFF
QFYGYKEFASKLFNITSKGEEKNFDVLSLFHRSDEAEDTLNENDISQFVQKISEAETNGLIRVNQKIM
EQLRGGLLIADEIHNVYNIQERNNYGIALQYVLDAFPPHQAPRAVFMSATPVTGSVMEYVDLLNLL
VPRHELPNGQPLQRQQLFDSSGHSVKWKKDALALVERLSTGRVSFLLDTNTNFYPERIFAGKMLSY
KDETLPYLHFIECPMSEYQLETLKQLGPDPKISSNAYSIYDMVFPNPKFSKQTEPKAYGLFNSTETPT
ALSMASTDWLLENGVQIIEPSRRAPFNVSGSFLSLQPPTHISGLAFYSGKYTQMMKDILSIIRQGRGKI
LIYHNRVRMSGVLILQEILQSNGILNEVSSPVGTTRCSICAAIRDEHTHSDHQFIPVRFTILHSEIEPAVR
ERSLALFNASSNLEGHQLRILIGSKVIVEGLNFQAVRYEMIMSLPLDIPRLIQVFGRVVRKNSHMELP
PSERNVTIYLYVSTTPDGGPELAKYAQKLKEYILIQEGDKALRKHAIDGFTNQIKIDKPMLESLPLSPS
ITPANVGATVLNTFEAYGYGEQEVKTISNIIISLFMARPVWTYSELWKAVSTPKLIQGMDNKLFSED
NFALALISLCYSKNQCKELWIQNRLCTIMHVPAKPEHLYVAAVLNHKKEPVLDIETYIRDFQPPAMH
SIRITKYLEHSQTKEPFQVLYEKFQKDFQDEPMEQVLIHYPASFHYTMLEALIIDNLAGMGALVEVY
KKFFIAFSKKDIQPFPDIFKIISHVPGDDNTLVGYATEDSVRLITSREDKTWHEIPLYMLNINVKRKEN
DIVIGYMESKGKALKFKIRPPIQVLKKNEITDIRMLNRGAVCETRGREEQQKIADQLGISLNLTKISAI
KLCLLIRNNLLQKEMEARNQPNGMQDGIRWFYLFNDKMPSLVHTSMATNFFIQPITEEAEAYYPPS
VITNKRKDLGVDVYCCSDLVLQPGLNIVRLHIKVACEHMGKKCGFKIMARSSMCTHERLLILANGI
GLIDPGYVGELMLKIINLGDTPVQIWAKECLVQLVAQGDHVPDHINILKRNQIFPLFAPTPRGEGRFG
STGEAGIMRTMKICKACSSCMVRTYVDGNIIFRCSCGESVQGDSQNLLVSSKVYHTGEMEDKYKIFI
KNAPFDPTNCQIKKDCPNCHLDYLTQICIGSQKIIILVCRCGYMSNRG
22. F1055L-E146L-I8L
(SEQ ID NO. 123)
MQETFKFLRCNSQGEAVEDKYSLETLKNHFVVRDEYNNLFRVFSSRDDFWEWEAAQPFEQKCFHE
VVFGFLPQRLKFDIDFPVNKSYSDDNVDDNDNVEDNVYDILDMIINVIMDVFYETYSLPYNINLTRE
QILLTDSIGLNKKRELKYSFHIILYTYSVLNNNEAKVFTSKVLENLPKHVYPFVDPQVNKSIQNFRIIG
SHKKGSMRVKMFNEELAEVFETSTTIKKSDTLIATPFETTCLPCIFTNVKETTPSSCDTIQQSELEEVL
KFAGTLCKNHCFLRVHKNLVLFKRTSPSYCEICKRMHDKDNTLILRVTGNKVYQHCRHDNKHSLL
MGSLSGTNNFVETYVDQVMTKSIEVHESILFEELPDTQKHIYDESSMREYERVPTLVVKAQMKIGKT
VQLRNYLQKYYGNNSISKQQTIRFVTFRQIFSKNIQSRLPNFTLYSEVTGDLDSYERVIIQVESLFRLT
STAEPVDLLILDEVESIFNQFNSGLHKYFAPSFAIFMWMLETANYVICLDANLGNRTYNILQRFRGD
VPIFFHWNQYKRAQHDTYYFTSSRETWLNNLLKDLLEDKKIVIPTNSLMEARLLQSFIQKKFPEKKI
GFYSSKSTAHERESHFNNVSYYWGLVDILIYTPTISAGVSYEDKRFDVLYGFFNNMSCDVETCCQM
LGRVRELKSKCYKICLQGKQNYYPETIEDIEMFTLQKRDTLFQTISNHQLSFTYSKETGRPIYYKTPY
YFILWLETMRIQHLSKNHFITRFINQIADTGAKVFILTGEKLETVKQYTSIKMEIKHQDYVNIASAETI
DANKALQIKQNLKEGITVDQQDLFAYEKYKLLEFYAWHGHKITPKFVEQYNSFMTKQNYTGRVQI
SRGKTVYESLTMLQTQELNFHQWAMQHAEHHDLQFNYSFQSHMYAIMLLTKCGFKCVQDPNILTN
EQLMTKLVDEFVQYDLSAISFEFKLKKPNKTDPQTILKFINKVLGLRYGLKIHHNKGNYYIKNTKAG
SLIPFVRQQIKQSPCVVSNLLPITETSSAKEETSPIKEETFTETMGGTTDFVLSITIVLVILIIIAFIWYNFT
GWSPFKYSKGNTVTFKTPDESSIAYMRFRNCVFTFTDPKGSLHSIDVTEVLNNMAKGFRDAQNPPSS
FTLGGHCQAPLNAFSFVLPGVNDRATVATADEAKKWENCDATLTGLQRIIMGNRLIKKDLKKCEY
YYGEQQNLKQIWRLLFNEPLGTYVVSSFLKKNYVVISFSCPTNTRIMHLRINICYDLYCINGEYYEKI
DDFIRLYPHIFYRPLYRYKS
23. B962L-H233R-H171R
(SEQ ID NO. 124)
MGKPILLEPGHLYNVPAEHKNDVPIHYIITWIKQRLPEFGGAIPTSLADRVLIIKSRTGSGKSTALPVH
VFRILRNENTHSFQKYLGRSVICTQPRVLTAVTLAKDIGASTHYPDMILGQTVGYQTKPLTEKPNRG
LIYATAGVLLAQLHTMTDDEIASRYAFMIIDEAHERALGIDLMLMYIKSMLQRMLQRGSIGALRIPF
VILTSATIDTHKYSTYFGIGKENIILVEGRQYGVETHWPLYNTNNYIKTACETALTIHKENIHDRPTEA
DILIFMPGMAEIRFLSMLLNNANMDLAKEKLPLMLILPIDSEAIAQENEAYLGLKAEIKNLWVKNPL
TAKVEKPLRRVIVSTVVAETGLTIETLKYVIDPGWNRSIETYYPEWAGGLITRPAAQSRIEQRKGRVG
RVFPGHFYPLYTKHVFEQIPVQQYPEIITEGPGAIFLSIVVETIKKNKEGVFKAEEIDMLDPPPTDALAS
AIERAIVAGLLTRGEKGLQLTQLGDIASRFSFLSIEEARMCFSGYFWQAAISDIATILAVVSVADKKLT
NLLDSKQRNGAMLAEAVLAGIPPFLQNIDNAYTNIHLLLADDLLEGLFIFEGFQHAIVYFINNKVNN
VAKHLREWCEKKMLKYSSMVQILARREDILNELAIVGLNPFHQWQNRLASANAETFLKRVCTLKQ
CMYEAYRLNCFCYDEHRLLYTGRNGIHFSYHDAVIKNPSCIVTPRIMLSPVSKQYMEWRLEPSFVSV
LDGFVNVDINFLLPRQEIPNILGGVENEEEEPPLPIQVFLHKYVKTHFHFSGKSFKELKMKPSQMIKFP
ETTLINMIPDIPKNVVQTYLEINVCHRYSFKRLIYCETFYTDMDDVQHENSVELIGLPMAAHHLTIND
FNKLYFILLKPDGFLIMYDLHQGQEAFWLHSLQDALGHHTIRRDMDFHTIPEWETIFKECGFTPIFSK
QPSEHELFIVFKKMILIASPFSLAHLEYLHTWHVTIKNIAQQHGLDIKVAIVVSTSHLNNFLPISGALNI
ECITFPSCGIKEIDLLWARIKLFQHYCAIGARLLWLVSADIRPPVSAWPAIADSLKKGADAVVIPYPSR
WNNLIPTVIKEIVVHQKKCLVAVDARHLDTDTQIVGAGMGCIVLTLKALMVRLSIGKQPVKILWPD
LHGTAEGIPLEGVEVGWFLNAYAFIKLNIRCLGADHIAQHLTMVVYDLLVSLSKESIDVLRFVEANL
AAFNQQYIFFNIQRKNSITTPLLITPQQEKISQIVEFLMDEYNKNNRRPSGPPREQPMFIPLLPYQQSSD
EQPMMPYQQPPGNDDQPYEQIYHKKHASQQVNTELNDYYQHILALGDEDKGMDSMLKLPEKAKR
DSDDEDDMFSIKN
24. C962R-MGF505-3R-C147L
(SEQ ID NO. 125)
MREESWEDHDTIQLTAQRKYLAEVQALETLLTRELSVFLTEPGSKKTNIINRITGKTYALPSTELLRL
YEHLEQCRKQGALMYFLERQGTYSGLMLDYDLKLNTNAVPPLEPPALSRLCHRIFVHIKNSSVLPE
GSHKIHFFFTLKPEVVQGKYGFHVLIPGLKLAASTKKSIIGSLQHDATVQKILHEQGVTNPESCLDPH
SASVPSLLYGSSKLNHKPYQLKTGFELVFDSSDPDYIPIHQIKNLESYNLVSELSLTNEQGSLVRPVYC
AADIAAEKEEEIPTEDHSLSILMLHDPEARYLHKILNLLPPEYYVEYPLWSNVVFALANTSANYRPLA
EWFSQKCPEKWNTGGKEKLEKLWNDASHHTEKKITKRSIMYWAHKHAPQQYKEIVEQGYFSILAE
YVYSYNGMLEHYMIAKVIYAMMGNKFVVDVDSNGKYVWFEFVLPGQPMNQGEIWKWRKEVNP
DELHIYISENFSRVMDRITEHIKYHLSQPHESNILNYYKKLLKAFERSKSKIFNDSFKKGVIRQAEFLF
RQRSFIQTLDTNPHLLGVGNGVLSIETIPAKLINHFHEHPIHQYTHICYVPFNPENPWTKLLLNALQDII
PELDARLWIMFYLSTAIFRGLKEALMLLWLGGGCNGKTFLMRLVAMVLGDHYASKLNISLLTSCRE
TAEKPNSAFMRLKGRGYGYFEETNKSEVLNTSRLKEMVNPGDVTARELNQKQESFQMTATMVAA
SNYNFIIDTTDHGTWRRLRHYRSKVKFCHNPDPSNPYEKKEDPRFIHEYIMDPDCQNAFFSILVYFW
EKLQKEYNGQIKKVFCPTIESETEAYRKSQDTLHRFITERVVESPSAETVYNLSEVVTAYAEWYNTN
INVKRHIALELSQELENSVLEKYLQWSPNKTRILKGCRILHKFETLQPGESYIGVSTAGTLLNTPICEP
KNKWWEWSPNPSAPPEKEASAPTPMSSSLQELCRKKLPDCILPEFFDDYVLQLLGLHWQDHGSLQR
IEKNQILVQQEPIHINEALKVAASEGNYEIVELLLSWEADPRYAVVGALESKYYDLVYKYYDQVKD
CHDILPLIQNPETFERCHELNSTCSLKCLFKHAVINDMLPILQKYTDYLDRWEYCSQMLFELACSKK
KYEMVVWIEGVLGVGKVTSLFTIAISNRDLQLYSLGYSIILENLYSCGQDPKFLLNHFLRDVSIKGLL
PFVIKTIEYGGSKEIAITLAKKYQHKHILKYFETWESMADNDNEDLIMDDLVEEYVETEEENLVDSE
EESEDKDEIVESPSICEGFVQASSQTLVIIPDNERITSNVLTTFEATRLVAVRAQQLAINGSTMLKKKY
SSPIDIAKQELFNRKIPLLVMRCIKVTPEGQKIVEIWNPREMGIPLLD
25. A859L-B318L-B169L
(SEQ ID NO. 126)
MCAGFYVAVHPWLEAQSLHKVGHTGNLAARLHDGSYTTCFTDEWKYCFTLETSTKKDAQKIEAG
VLYCAQFFRVKNKELVCLLPEKIKQLAEDVANCLDISYTLCDSPAYEMNDSTIVVEPSLPSDPLISKE
KLRHLVITPVEDEHFADDVLFFSTDETRTAIEDRLYQKEAANMGYQELRRSGRAILQMACRCGKTR
VAYLILSNYLQGKVLYLVPGLSLLRQTLEKLYQYGISLKNVLLVGSDQTRIVLNHDNIEMTTNPVFI
AKRIREAPSLLVIATYQSSTLLVDDFDLIISDECHRICGEWETRPFTHVLLNFKKGHRLFLTATPRYDT
PLSMKNRELFGGVAFRYYLREGIEAGYVNDFELQMVAAPKLAHQPSTKEETTKQIIVKQIIMALAYL
KTNIPAPKMLVFTRDIKQAKELYAALVDQGVYALIAHSTLPRQVILKTFTEFCSSKEPVILLNCRLFQ
EGVEVPELNAVFFAAPRHSPRDIIQSICRPLNKQVQKPHATIFLPLEVNTENVCLDRFSSIIPFADALAS
EDPRFYEHLLNPSEVAYPINWIGAHGSVSELLQLARHAIRYGTQGKIDRLTRSERLPWKAAFAELKR
TVEICCRYPKINDGFHFGGATLRFDTWYKWVIKSYLQYKNKEPSSLEPYQVSDLESLQDWTTRGVG
GPYPWEESMAFLETWLAQNKGELVAIDIHQGGWIGLDATPMERLSGVLTTVSQRDGRSYGKNKKL
RPKKGFMIPPQQAQDLDRIFGKHNLKWRKDRVNGFLKEDEHGNYTGEPTCIQEAYRTFKEYVKTNP
EYIEKYWPGYAKGKHKHQELPHIWEKGLAPPRYKAFKDGNKQLIQRSPKKKDIKNMLHLIYISIIVV
LIIILISYTRKPKYFRITAPRSVALFHGIHPLNPKNYKTFSEEFETILNNAIEDGDFKGQLTEPCSYALRG
GKYIRPIILMEIVRACQLQHSFGAPIYPAEAALAAEYFHVASLIIDDMPSFDNDVKRRNKDTVWARF
GVAKAQMSALALTMQGFQNICRQIDWIKEHCPRFPDPNQLGALLCTFVSHSLNSAGSGQLVDTPEK
TIPFFKIAFIMGWVLGTGSVEDIGMIERAAHCFGHAFQLADDIKDHDTDTGWNYAKIHGKQKTFDD
VAQSLQECKKILHGKKIFTSIWNEIFQKVINVALGTMNVDFIAGINNLGEKIYTCEPFKTSFQNPFIVA
LIITAVVLVVFFAICNPPVDKKRKTKTAIYVYICIVALLFLHYYVLNHQLNDIYNKSNMDVIVSSIHD
KYKGGDEIIPPISPPSVSNELEEDQPKKIPAGPKPADSKPVSLPDSKPLVPLQEVIMPSQYNN
26. C717R-H359L-F317L
(SEQ ID NO. 127)
MTKLAQWMFEQYVKDLNLKNRGSPSFRKWLTLQPSLLRYSGVMRANAFDILKYGYPMQQSGYTV
ATLEIHFKNIRSSFANIYWNRDSEEPEYVCCCATYQSFIDGEYRYRFVWYQPFIEAYNAIEAALDPLE
TTILNLIAARDLDFVVHIFPYNKGHEDYLASTQLILKIFIATLLMDILRIKDNTLDVHLNSDYIIVMERL
WPHIKDAIEHFFEAHKDLLGYLIAFRNGGNFAGSLRPSCGQKIVPLTIREVLQMNDINLAVWREVFI
MQECSDLVINGIAPCFPIFNTWTYLQGINQIFFENTSLQEKFKKDFIARELSKEIIKGQKTLNDKEFKK
LSLHQIQYMESFLLMSDVAIMITTEYVGYTLQSLPGIISRSSYLSPIVKNILMDEDSFMSLLFDLCYGA
YVLHKKENVIHADLHLNNMTYYHFNPTSFTDRNKPGKYTLKVKNPVIAFITGPKVETETYVFKHID
GFGCIIDFSRAIMGPNHAIKLERQYGLAFVNTFYRNQSEHILKVLRYYFPEMLTNRENEIQGVILSNF
NFFFNSITAIDFYAIARNLRSMLSLDYLHTSEVKRNVEISQTFLDTCQFLEEKAVEFLFKNLHTVLSGK
PVEKTAGDVLLPIVFKKFLYPNIPKNILRSFTVIDVYNYNMKRYSGKAIQTFPPWAQTKEILTHAEGR
TFEDIFPRGELVFKKAYAENNHLDKILQRIREQLANENLMEKIFQNVEIKPFLIDFSNLFIKNAAKKLF
QLEEQLPLVPVNVVMDFKGISRAAVHGLSRVLQDEIPNYMLDIKPGGYKIEDSTDLFMTEQFIRNRI
NFIPIYAKNETLVFALRSLNNSCEVKTIYSRDLIQVAGPKLKYPIFNPTFEIGFLQPGKSLIIEDIYIKKGI
GRKHAAFNLAVKTHFSHLDIEQYPTDKKEYMALSGYKQSSMTSDPRHHRLGLCFPAVPLPHINQAV
RTYLKNACRIIIGRIQSIQKIYENFEEPQPELVLFSMDEEKTKAIMKDETHTIGNLLKTYIYEMIPDISF
VGYQCVPHKQEMVLTIIHKASQEDLITLLEKSIQNIIQTFQILEKNVDELIAMVETQMDKLGFLLNHI
GKQVTTKVLSNAHITQTMKEIILENHSVDGGAAKNVSKGKSSPKEKKHWTEFESWEQLSKSKRSFK
EYWAERNEIVNTLLLNWDNVRGAIKKFLDDDREWCGRINMINGVPEIVEIIPSPYRAGENIYFGSEA
MMPADIYSRVANKPAMFVFHTHPNLGSCCGGMPSICDISTTLRYLLMGWTAGHLIISSNQVGMLTV
DKRIIVDLWANENPRWLMAQKILDIFMMLTSRRSLVNPWTLRDLKKILQDYGIEYIIFPSNDFFIYED
ERLLMFSKKWTNFFTLHELLDDLETTETKASSTT
27. MGF505-10R-B475L-MGF360-14L
(SEQ ID NO. 128)
MFSLQELCRKNIYILPYPLAKHVLQQLGLYWKGHGSLQRIGDDHVLLQQDLIFSINEALRMAGEEG
NNEVVKLLLLWEGNLHYAIIGALEGDRYDLIHKYYDQIGDCHKILPLIQDPQIFEKCHELSNSCNIRC
LLEHAVKFIDMLSILQKHKEQIRLHMALTQILFELACHERKNDIIRWIGYSLHIHHLETIFDVAFAHKN
LSLYVLGYELLMHKVNTEAAYIELPNLLSYHLRTAAAGGLLNFMLETIKHGGYLDKTVLSAAIRYK
HRKIVAHFIHQVPRKTVKKLLLYAVQARAPKKTLNLLLSSLNYSVHTITKQLVHNVVIYSSTLIVKLL
LMRRKNKLNLVDAVLARLVKYSTYTDIVQFMGEFSVSPERVIKMAARESRTFLIEMISKAAWGNHP
QTLIHHLKQLTNTMKPQSGKDHIIYTIHYIYLNSNMLVAEEEKNIFKLAKFYANHNAVNRFKQICED
YYILDARFKTLILECFEIAVQKNYPRIANIVDDYIRFLFYRGNITEEEIREAYSLKDAEVYVDLKWLQ
QGEMVMDQEESHVISIFETLGAYFINIFYNFLYKNALYKKHSIVTEYQYQVKGYILGVKQNKKLYE
KMLDSFYKYFCNITQINSKTLNFSNFVTTIVDSFIPKEYSQSISLEKKESILELLLCDYISNLGTFITTEK
MLPFIIKNRKENYHKVTKEMQDYSLTFLLKKRMELYNKFLRKQAYVEPETELEETYARLSSYNRSL
LHQIEELTSENKSLLADLSTLRKKYEKRQSEYRRLVQLLYQQIQRSSTSKSSYPLTKFIETLPSEHFSN
EEYQKETPADQKEVVEMELLRKQELLTSQELTSKSPNNYPVPHSRTIVSKPLDNYPVPRSRTTTKIDF
DNSLQNQELHTKNGFSEKDIVEFGQDKPEEENILAIDQDKPEEETTLAIKQDISEEDNIFAIDQDKPEFN
QDTPEFKEAVLDIKENILEEENQDEPIVQNPFLENFWKPEQKTFNQSGLFEESSNFSNDWSGGDVTLN
FSMLSLQTLAKKVVACNYLSSDYDYTLQRFGLWWDLGPIHLCNNCKQVFSYKHLQCFSEDDLCLE
AALVKAVKSDNLELIRLFVDWGANPEYGLIRVPAVYLKRLCAELGGLTPVSEPRLLEILKEVARLKS
CAGVLLGYDMFCHNPLLETVTRTTLDTVTYTCSNIPLTGDTAHHLLTKFWFALALRHNFTKAIHYF
YKRHKNHLYWRVACSLYFNNIFDIHELCREKEICISPNLMMKFACLREKNYAAIYYCHRLGASLDY
GMNLSIYNNNTLNMFFCIDLGAADFDRAQLIAFIKAYMYNLSNIFLVKQLFSRDVTLVLDVTEPQEIY
DMLKTYTSKNLKRAEEYLTAHPEIIVID
28. MGF505-2R-B407L-MGF360-13L-E111R
(SEQ ID NO. 129)
MFSLQDLCRKHLFILPDVFGEHVLQRLGLYWRCHGSLQRIGDDHILIRRDLILSTNEALRMAGEEGN
NEVVKLLLLWKGNLHYAVIGALQGDQYDLIHKYENQIGDFHFILPLIQDANTFEKCHALERFCGVS
CLLKHATKYNMLPILQKYQEELSMRAYLHETLFELACLWQRYDVLKWIEQTIHVYDLKIMFNIAIS
KRDLTMYSLGYIFLFDRGNTEATLLTQHLKKTAAKGLLHFVLETLKYGGNIDTVLTQAVKYNIIRK
LLDYFLRQLPRKHIEKLLLLAVQEKASKKTLNLLLSHLNYSVKRIKKLPRYVIEYESTLVIKILLKKR
VNLIDAMLEKMVRYFSATKVRTIMDELSISPERVIKMAIQKMRTDIVIHTSYVWEDDLERLTRLKN
MVYTIKYEHGKKMLIKVMHGIYKNLLYGEREKVMFYLAKLYVAQNAATQFRDICKDCYKLDVAR
FKPRFKQLILDCLEIITKKSCYSILEILEKHIISLFTMKVMTEEEKNLCLEILYKVIHYKTIQCMEDTTFL
EGANLAGITTLMNNLHINEQANLEELEKQVMGKQQSFPTDHFDEELNGLAKSLGINFNDPEFSLDSP
HSIISKKPSGRGGDKVHGGIRRDSVCTDSICSDSVCSGSIRSGSIRSGSIRNGSIRSGSVRDGSIRNGSVR
SGKTRRGLACNSSSRNDRGYSLSTHRKKYAESEASQKTAISKRDRKNHYAESEYSEKSIKPSTKQVD
RLINHLRSNGDPNSFYKKDFIDYERKTKLVKLEKINMLLTYLGNEQISTDDIKIPTIDSSMQEIDDVIE
MLTLRNVGIRYSSIAEEILIGLARGLEIVFDGTREIPFLNYRPDYTGLHNTFMIKLFKMRYETSQVVGN
LVQNMSPLSKICLELGPSLLLYPALIRTKHKASEDLYNLLQKGPEDPFTAYNEIHETLKKNNKMSLPL
SLQTLVKKTIASQCLSIDEHCILKYCGLWWHDAPLKLCMDRGRIQIKSGFLGEDIDLRVALIIAVKEN
NYSLIKLFTEWGANINYGLLSINTKHIRELCRQLGAKETLEDNDIFRIFTRIMHNKTSGSIILCHEIFMN
NPILENKFVIQLRGLIYKRLWGLIEIKETDELNGLLVKYWYAKAVQYDCKDAICFLDEKYTDLNEW
RLKCLLYYNKIYELHEMYHKENIQIDVHDMICLASTKDNNPLTIYYCYALGGNINQAMLTSVQYYN
IGNIFFCIDLGGNAFEEGRAIAEQKGYNFLSHSLALDIYSSDASLPLNLKDPEETSSLLKDYKSKNLSII
WEYSHNILMSFSECPLVISACKKFLQKRITIENEALINALITALAQTSTLNDLCLLPIQTYLLSYKNAFE
WIHFVCIAITTILDNKYNWKDCTVDINYIFLHVTYIYNIKTKEYLDYCS
29. K421R-EP424R-D339L-S183L
(SEQ ID NO. 130)
MYTHVDVVGIAEASAALYVQKDRDRYLDVLTTIENFIYQHKCIITGESAHLLFLKKNIYLYEFYSNN
VAEHSKALATLLYKLDPEYLTRYTVLITKIPNHWYVINVDQREFVRLYAIPAVKQHLPIPILPFYCTS
ALTQQELFCLGPELQLIQIYSKLCNPNFVEEWPTLLDYEKSMRMLFLEQFPQRLEMTGGKKEEKEK
HESIIKKIILEMVSTRQRIVVGGYIQKNLYNHVLKNRNRLQLITSLNIYEEKDIIQQFCDSNGLKIKIRIN
NPLLPTNPELRRLTIYFNHNNDDDQSYLIVDMYNTGSYELVPTNQINTLDGSFLIGTPFVQARFLLVEI
WVLMLIAQQTKKDTKKIIQFFINQYEMLMNSPWPSMEALFPSSSKRYLGNYVDPNALIKWAQLKLK
RIPPFYPGKPDEESCMSNYYYYYGGGRYDWLKTVEPTNFLKIGLPYQAFIPLHLQHQATTPPSILEKF
KRADILLNEVKAEMDPLMLQPETEKKLFQILSSIDMFKGLRKKVEFTYNAQIVTNAWLKMYELLNT
MNFNNTSQAFCNCELPGGFISAINHFNYTMMHYPTFNWVASSLYPSSETDALEDHYGLYQCNPDN
WLMQSPLLKKNIDYNNGDVTIASNVKNLALRATQRLTPIHLYTADGGINVGFIDYNKQEELNLKLH
FGQALTGLLSLSKGGNMILKHYTLNHAFTLSLICVFSHFFEELYITKPTSSRPTNSETYIVGKNRLRLF
TPKEEQVLLKRLEFFNDTPLVDLSLYQNLLESVYFAVETIHLKQQIEFLNFGMKCYRHFYNKIKLLN
DYLAPKKKIFQDRWRVLNKLYVLEKKHKLKLCASMIDQKIFETTLNIDDPTNFCTNVEAHLLKELE
NIYVGKCFKNSFILNITGVIQRSPCFIMRTNNSGRGYMHVRFSAVVSYLNAFDLIAAVKIIKNDSNIIL
GESLLTEPVTIVIPSSESQNNVAEVGQIVPVQLANSSVYYIPGRQQASATGSIFIPKHTFSVYHVQEEL
TQEQALNLTKLVNIIEMLLESRSKKDFKQICFFEKLYYTYSISSDEILDLKIWKGPKGKEMSRLKPCN
VLSFLYDALKNKNSSLGFWARPPNLLKSSPLAYQQDQNSFNATELPIICSAEVMFVTLLKEIINYLQFI
NDLCDTFNNEQLIKRHENIWMLIEQRKIGHDFMSVVVGGVEYSLNNWARYEIKRRAAELESVNYYP
HCEYIMPEDIVVSILGSKPNCPFLEALKRFHDFLKKRRIIFKGEYLVIPWMGAQDVADMIHHVENRIN
LDHLEDLAHMLKLITYHRSFDTCINQAFEHLYAFKFPDANIETHELKHIRQLEKKMYGYILRLEKLQ
TVLTFYIEFLLKQV
30. EP296R-B263R-C257L-I243L-A179L-B117L
(SEQ ID NO. 131)
MFGAFVSHRLWSDSGCTTTCITNSIANYVAFGEQIGFPFKSAQVFIAGPRKAVINIQEDDKVELLKMI
VKHNLWVVAHGTYLDVPWSRKSAFVTHFIQQELLICKEVGIKGLVLHLGAVEPELIMEGLKKIKPV
EGVVIYLETPHNKHHTYKYSTIEQIKELFLRIRNTRLKQIGLCIDTAHIWSSGVNISSYNDAGQWLRSL
ENIHSVIPPSHIMFHLNDAATECGSGIDRHASLFEGMIWKSYSHKIKQSGLYCFVEYVTRHQCPAILE
RNLGSSMQLQTALTAEFTTLKSLLKMEDETELCFRSNKVTRLEMFVCTYGGKITSLACSHMELIKM
LQIAEPVKALNCNFGHQCLPGYESLIKTPKKTKNMLRRPRKTEGDGTCFNSAIEASILFKDKMYKLK
CFPSTGEIQVPGVIFPDFEDGKNIIQQWVDFLQHQPIEKKIQIIEFKTIMINFKFQINPVSPRVIIHLKKFA
ALLEHIPTPYPIREIKPPLEDSKVSAKFMVSPGKKVRINVFLKGKINILGCNTKESAETIYTFLKDLISV
HWQEILCVLPVPDMYSVCDVVRDAVAQSHLCACPNDKLPQCKGVTKAPPKCSVFHVAKLQDTKF
KWKYTLDPLKAQKLSQIDKDIEKDAITLKLIYGIELSPEDLEWWKMQRCLINKKTGAKGGQFANKY
LERQDLELLGYSPTPIIGGDFMFTALPDKVLRTIPVAWDRFLNPAMMIFFLIILLCVILGIFYVLVRNTL
RRKQKSKQHQMEIKRFIKEKEQDPYIHTSFESWPADPNKEWKDLIPVYEAQGYCMADYRKKLGMP
PGPNCMKMHIARDSIVFLLNKYLQNTILTNKIEQECFLQADTPKKYLQYIKPFLINCMTKNITTDLVM
KDSKRLEPYIILEMRDIIQMMFFRTLQKHMFFKEHTDLCTEYAQKIEASCYHYTYQQQEKTFLEEYS
TRCGTINHIINCEKKSHQQQDNDALNKLISGELKPEAIGSMTFAELCPSAALKEKTEITLRSQQKVAE
KTSQLYKCPNCKQRMCTYREVQIRALDEPSTIFCTCKKCGHEFIGMEGEELIYHNIINEILVGYIKYYI
NDISEHELSPYQQQIKKILTYYDECLNKQVTITFSLTSVQEIKTQFTGVVTELFKDLINWGRICGFIVFS
AKMAKYCKDANNHLESTVITTAYNFMKHNLLPWMISHGGQEEFLAFSLHSDMYSVIFNIKYFLSKF
CNHMFFRSCVQLLRNCNLIMGYTIQLDKDGDYCWDEDPTHHDPYMQANATSHVATSYATTSHAA
TPHAAAHHTFHEPFIKLNLTDKNIFNGLGFILIVIFIYLLLITLQQMLTRHIYNTVQHCVKAHLDSKNL
Q

Such multicistronic cassettes can be administered to animals as described above.

What is claimed is:

1. An immunogenic composition comprising a chimeric gene comprising at least one nucleic acid sequence encoding for a sequence having at least 80% sequence homology with any one or more of SEQ ID NOS: 1-101, and any combination thereof.

2. The immunogenic composition of claim 1, wherein the chimeric gene includes at least two of said nucleic acid sequences.

3. The immunogenic composition of claim 2, wherein said chimeric gene includes a self-cleaving peptide linker between each of the at least two nucleic acid sequences.

4. The immunogenic composition of claim 1, wherein said immunogenic composition is in the form of a multicistronic cassette.

5. The immunogenic composition of claim 1, wherein said chimeric gene is inserted into a vector.

6. The immunogenic composition of claim 5, wherein said vector is selected from the group consisting of an adenovirus, baculovirus, or lentivirus vector.

7. The immunogenic composition of claim 6, wherein said vector is a single-cycle replicon adenovirus or an attenuated bovine parainfluenza virus type 3 genotype c (BPIV3c).

8. The immunogenic composition of claim 1, wherein said chimeric gene is selected from the group consisting of SEQ ID NOS. 102-131.

9. The immunogenic composition of claim 1, further comprising an antigen from another disease-causing organism.

10. The immunogenic composition of claim 9, wherein said another disease-causing organism is selected from the group consisting of Actinobacillus pleuropneumonia; Adenovirus; Alphavirus such as Eastern equine encephalomyelitis viruses; Bordetella bronchiseptica; Brachyspira spp., preferably B. hyodyentheriae; B. piosicoli, Brucella suis, preferably biovars 1, 2, and 3; Classical swine fever virus; Clostridium spp., preferably Cl. difficile, Cl. perfringens types A, B, and C, Cl. novyi, Cl. septicum, Cl. tetani; Coronavirus, preferably Porcine Respiratory Corona virus; Eperythrozoonosis suis; Erysipelothrix rhsiopathiae; Escherichia coli; Haemophilus parasuis, preferably subtypes 1, 7 and 14: Hemagglutinating encephalomyelitis virus; Japanese Encephalitis Virus; Lawsonia intracellularis; Leptospira spp.; preferably Leptospira australis; Leptospira canicola; Leptospira grippotyphosa; Leptospira icterohaemorrhagicae; and Leptospira interrogans; Leptospira pomona; Leptospira tarassovi; Mycobacterium spp. preferably M. avium; M. intracellulare; and M. bovis; Mycoplasma hyopneumoniae (M hyo); Pasteurella multocida; Porcine circovirus; Porcine cytomegalovirus; Porcine Parvovirus; Porcine Reproductive and Respiratory Syndrome (PRRS) Virus; Pseudorabies virus; Rotavirus; Salmonella spp.; preferably S. thyhimurium; and S. choleraesuis; Staph. hyicus; Staphylococcus spp. preferably Streptococcus spp., preferably Strep. suis; Swine herpes virus; Swine Influenza Virus; Swine pox virus; Swine pox virus; Vesicular stomatitis virus; Virus of vesicular exanthema of swine; Leptospira hardjo; and/or Mycoplasma hyosynoviae.

11. The immunogenic composition of claim 1, further comprising at least one pharmaceutical-acceptable carrier.

12. A immunogenic composition comprising at least one recombinant sequence selected from the group consisting of sequences having at least 80% sequence identity to one of SEQ ID NOS. 1-101.

13. The immunogenic composition of claim 12, further comprising an antigen from another disease-causing organism.

14. The immunogenic composition of claim 13, wherein said another disease-causing organism is selected from the group consisting of Actinobacillus pleuropneumonia; Adenovirus; Alphavirus such as Eastern equine encephalomyelitis viruses; Bordetella bronchiseptica; Brachyspira spp., preferably B. hyodyentheriae; B. piosicoli, Brucella suis, preferably biovars 1, 2, and 3; Classical swine fever virus; Clostridium spp., preferably Cl. difficile, Cl. perfringens types A, B, and C, Cl. novyi, Cl. septicum, Cl. tetani; Coronavirus, preferably Porcine Respiratory Corona virus; Eperythrozoonosis suis; Erysipelothrix rhsiopathiae; Escherichia coli; Haemophilus parasuis, preferably subtypes 1, 7 and 14: Hemagglutinating encephalomyelitis virus; Japanese Encephalitis Virus; Lawsonia intracellularis; Leptospira spp.; preferably Leptospira australis; Leptospira canicola; Leptospira grippotyphosa; Leptospira icterohaemorrhagicae; and Leptospira interrogans; Leptospira pomona; Leptospira tarassovi; Mycobacterium spp. preferably M. avium; M. intracellulare; and M. bovis; Mycoplasma hyopneumoniae (M hyo); Pasteurella multocida; Porcine circovirus; Porcine cytomegalovirus; Porcine Parvovirus; Porcine Reproductive and Respiratory Syndrome (PRRS) Virus; Pseudorabies virus; Rotavirus; Salmonella spp.; preferably S. thyhimurium; and S. choleraesuis; Staph. hyicus; Staphylococcus spp. preferably Streptococcus spp., preferably Strep. suis; Swine herpes virus; Swine Influenza Virus; Swine pox virus; Swine pox virus; Vesicular stomatitis virus; Virus of vesicular exanthema of swine; Leptospira hardjo; and/or Mycoplasma hyosynoviae.

15. The immunogenic composition of claim 12, further comprising at least one pharmaceutical-acceptable carrier.
